(19) United States
(12) Patent Application Publication
(10) Pub. No.: US 2026/0244102 A1
(43) Pub. Date: Aug. 20, 2026

(54) POLYMERIZABLE COMPOUNDS, PHOTORESIST COMPOSITIONS, AND PATTERN FORMATION METHODS

(71) Applicant: DuPont Electronic Materials International, LLC, Marlborough, MA (US)

(72) Inventors: Emad Aqad, Northborough, MA (US); Paul J. LaBeaume, Auburn, MA (US); Conner A. Hoelzel, Marlborough, MA (US); Vageesha Warnajith Liyana Gunawardana, Marlborough, MA (US); Tomas Marangoni, Orono, ME (US); Mingqi Li, Shrewsbury, MA (US); James F. Cameron, Brookline, MA (US); Edon Vitaku, Marlborough, MA (US); Kenneth Hernandez, Ashland, MA (US)

(21) Appl. No.: 19/529,548

(22) Filed: Feb. 4, 2026

Related U.S. Application Data
(60) Provisional application No. 63/758,733, filed on Feb. 14, 2025.

(51) Int. Cl. G03F 7/038; C07C 39/20; C07C 69/54; C07C 309/60; C07C 381/12; C08F 20/68

(52) U.S. Cl. CPC G03F 7/0388; C07C 39/20; C07C 69/54; C07C 309/60; C07C 381/12; C08F 20/68; C07C 2601/08

(57) ABSTRACT

A polymerizable compound including an anion; a cation; and a polymerizable group bonded to the anion or the cation wherein the anion comprises: (i) a monocyclic or polycyclic C₃₋₆₀ aromatic group that comprises a first substituent group, and (ii) an anion stabilizing group, wherein the first substituent group comprises an anion group, wherein the anion group is selected from a sulfonate anion group, a sulfinate anion group, a sulfonimide anion group, a sulfamate anion group, or a sulfonamide anion group, wherein the anion stabilizing group is configured to form an intramolecular non-covalent bond with the anion group, and wherein the first substituent group further comprises the anion stabilizing group, or the monocyclic or polycyclic C₃₋₆₀ aromatic group further comprises a second substituent group, wherein the second substituent group comprises the anion stabilizing group.

### CROSS-REFERENCE TO RELATED APPLICATIONS

This application claims priority to and the benefit of U.S. Provisional Application Ser. No. 63/758,733, filed on Feb. 14, 2025, in the United States Patent and Trademark Office, the entire contents of which are incorporated herein by reference.

### FIELD

The present invention relates to polymerizable compounds, photoresist compositions, and to pattern formation methods using such photoresist compositions. The invention finds particular applicability in lithographic applications in the semiconductor manufacturing industry.

### BACKGROUND

Photoresist compositions are photosensitive materials used to transfer a pattern to one or more underlying layers, such as a metal, semiconductor, or dielectric layer disposed on a substrate. Positive-tone chemically amplified photoresist compositions are conventionally used for high-resolution processing. Such resist compositions typically include a polymer having acid-labile groups and a photoacid generator (PAG). A layer of the photoresist composition is pattern-wise exposed to activating radiation and the PAG generates an acid in the exposed regions. During post-exposure baking, the acid causes cleavage of the polymer's acid-labile groups and a resulting polarity-switch of the polymer in the exposed regions. This creates a difference in solubility characteristics between exposed and unexposed regions of the photoresist layer in a developer solution. In a positive tone development (PTD) process, exposed regions of the photoresist layer become soluble in a developer, typically an aqueous base developer, and are removed from the substrate surface while unexposed regions remain on the substrate to form a positive relief image. Alternatively, in a negative tone development (NTD) process, unexposed regions of the photoresist layer can be removed with an organic solvent developer, typically n-butyl acetate, while the exposed regions remain on the substrate to form a negative relief image. The resulting relief image permits selective processing of the substrate.

A property of the photoresist composition that can directly impact semiconductor manufacturing cost is photosensitivity, i.e., sensitivity to the activating radiation generated by the exposure tool, with a higher sensitivity corresponding to a higher process throughput for a given feature size. For increasing photosensitivity, it is desirable that the PAG generates an acid of sufficiently high strength to cleave the acid-labile groups on the polymer. For this purpose, typical are ionic PAG compounds having a photoactive cation and an anion with a fluorinated sulfonate group, wherein fluorine atoms and/or fluoroalkyl groups are in close proximity to the sulfonate group, typically bonded as substituents to one or more alkylene carbon atoms bonded to the sulfonate anion group. Upon exposure to activating radiation, the photoactive cation undergoes a cascade of photochemical and chemical processes that leads to the formation of a fluorinated sulfonic acid. Certain fluorinated PAGs in this class of compounds, while allowing for photoacids high in acidity, are becoming of increased interest by the semiconductor manufacturing industry and governmental regulatory bodies for replacement with more sustainable alternatives.

Examples of existing fluorine-free PAGs include p-toluenesulfonate anions and camphorsulfonic anions. These anions, however, have relatively low acid-dissociation constants (e.g., about 18 orders of magnitude smaller than that of tris(trifluoromethylsulfonyl)methane), which limits their usefulness in photoresists requiring a higher-strength photoacid. It would therefore be desirable to have a photoresist composition containing an ionic photoacid generator compound that generates a sulfonic acid of sufficient strength that does not rely on particular fluorine substitution for the increased acidity.

There is a continuing need for photoresist compositions that address one or more problems associated with the state of the art, and for patterning methods using such photoresist compositions.

### SUMMARY

An aspect provides a polymerizable compound including an anion; a cation; and a polymerizable group bonded to the anion or the cation wherein the anion comprises: (i) a monocyclic or polycyclic C₃₋₆₀ aromatic group that comprises a first substituent group, and (ii) an anion stabilizing group, wherein the first substituent group comprises an anion group, wherein the anion group is selected from a sulfonate anion group, a sulfinate anion group, a sulfonimide anion group, a sulfamate anion group, or a sulfonamide anion group, wherein the anion stabilizing group is configured to form an intramolecular non-covalent bond with the anion group, and wherein the first substituent group further comprises the anion stabilizing group, or the monocyclic or polycyclic C₃₋₆₀ aromatic group further comprises a second substituent group, wherein the second substituent group comprises the anion stabilizing group.

Another aspect provides a photoresist composition including the polymerizable compound, or a polymer comprising a repeating unit derived from the polymerizable compound; and a solvent.

Still another aspect provides a patterning method comprising applying a layer of the photoresist composition on a substrate to provide a photoresist composition layer; pattern-wise exposing the photoresist composition layer to activating radiation to provide an exposed photoresist composition layer; and developing the exposed photoresist composition layer to provide a resist relief image.

### DETAILED DESCRIPTION

Reference will now be made in detail to exemplary embodiments, examples of which are illustrated in the present description. In this regard, the present exemplary embodiments may have different forms and should not be construed as being limited to the descriptions set forth herein. Accordingly, the exemplary embodiments are merely described below, by referring to the figures, to explain aspects of the present description. As used herein, the term “and/or” includes any and all combinations of one or more of the associated listed items. Expressions such as “at least one of,” when preceding a list of elements, modify the entire list of elements and do not modify the individual elements of the list.

As used herein, the terms “a,” “an,” and “the” do not denote a limitation of quantity and are to be construed to cover both the singular and the plural, unless otherwise indicated herein or clearly contradicted by context. “Or” means “and/or” unless clearly indicated otherwise. The modifier “about” used in connection with a quantity is inclusive of the stated value and has the meaning dictated by the context (e.g., includes the degree of error associated with measurement of the particular quantity). All ranges disclosed herein are inclusive of the endpoints, and the endpoints are independently combinable with each other. The suffix “(s)” is intended to include both the singular and the plural of the term that it modifies, thereby including at least one of that term. “Optional” or “optionally” means that the subsequently described event or circumstance can or cannot occur, and that the description includes instances where the event occurs and instances where it does not. The terms “first,” “second,” and the like, herein do not denote an order, quantity, or importance, but rather are used to distinguish one element from another. When an element is referred to as being “on” another element, it may be directly in contact with the other element or intervening elements may be present therebetween. In contrast, when an element is referred to as being “directly on” another element, there are no intervening elements present. It is to be understood that the described components, elements, limitations, and/or features of aspects may be combined in any suitable manner in the various aspects.

Unless otherwise defined, all terms (including technical and scientific terms) used herein have the same meaning as commonly understood by one of ordinary skill in the art to which this invention belongs. It will be further understood that terms, such as those defined in commonly used dictionaries, should be interpreted as having a meaning that is consistent with their meaning in the context of the relevant art and the present disclosure, and will not be interpreted in an idealized or overly formal sense unless expressly so defined herein.

As used herein, “actinic rays” or “radiation” means, for example, a bright line spectrum of a mercury lamp, far ultraviolet rays represented by an excimer laser, extreme ultraviolet rays (EUV light), X-rays, particle rays such as electron beams and ion beams, or the like. In addition, in the present invention, “light” means actinic rays or radiation. The krypton fluoride laser (KrF laser) is a particular type of excimer laser, which is sometimes referred to as an exciplex laser. “Excimer” is short for “excited dimer,” while “exciplex” is short for “excited complex.” An excimer laser uses a mixture of a noble gas (argon, krypton, or xenon) and a halogen gas (fluorine or chlorine), which under suitable conditions of electrical stimulation and high pressure, emits coherent stimulated radiation (laser light) in the ultraviolet range. Furthermore, “exposure” in the present specification includes, unless otherwise specified, not only exposure by a mercury lamp, far ultraviolet rays represented by an excimer laser, X-rays, extreme ultraviolet rays (EUV light), or the like, but also writing by particle rays such as electron beams and ion beams.

As used herein, the term “hydrocarbon” refers to an organic compound or group having at least one carbon atom and at least one hydrogen atom; “alkyl” refers to a straight or branched chain saturated hydrocarbon group having the specified number of carbon atoms and having a valence of one; “alkylene” refers to an alkyl group having a valence of two; “hydroxyalkyl” refers to an alkyl group substituted with at least one hydroxyl group (—OH); “alkoxy” refers to “alkyl-O—”; “carboxyl” and “carboxylic acid group” refer to a group having the formula “—C(O)—OH”; “cycloalkyl” refers to a monovalent group having one or more saturated rings in which all ring members are carbon; “cycloalkylene” refers to a cycloalkyl group having a valence of two; “alkenyl” refers to a straight or branched chain, monovalent hydrocarbon group having at least one carbon-carbon double bond; “alkenoxy” refers to “alkenyl-O—”; “alkenylene” refers to an alkenyl group having a valence of two; “cycloalkenyl” refers to a non-aromatic cyclic divalent hydrocarbon group having at least three carbon atoms, with at least one carbon-carbon double bond; “alkynyl” refers to a monovalent hydrocarbon group having at least one carbon-carbon triple bond; the term “aromatic group” refers to a monocyclic or polycyclic aromatic ring system that satisfies Huckel's Rule (4n+2π electrons) and includes carbon atoms in the ring; the term “heteroaromatic group” refers to an aromatic group that includes one or more heteroatoms (e.g., 1-4 heteroatoms) selected from N, O, and S instead of a carbon atom in the ring; “aryl” refers to a monovalent monocyclic or polycyclic aromatic ring system where every ring member is carbon, and may include a group with an aromatic ring fused to at least one cycloalkyl or heterocycloalkyl ring; “arylene” refers to an aryl group having a valence of two; “alkylaryl” refers to an aryl group that has been substituted with an alkyl group; “arylalkyl” refers to an alkyl group that has been substituted with an aryl group; “aryloxy” refers to “aryl-O—”; and “arylthio” refers to “aryl-S—”.

The prefix “hetero” means that the compound or group includes at least one member that is a heteroatom (e.g., 1, 2, 3, or 4 or more heteroatom(s)) instead of a carbon atom, wherein the heteroatom(s) is each independently N, O, S, Si, or P; “heteroatom-containing group” refers to a substituent group that includes at least one heteroatom; “heteroalkyl” refers to an alkyl group having at least one heteroatom instead of carbon; “heterocycloalkyl” refers to a cycloalkyl group having 1-4 heteroatoms as ring members instead of carbon; “heterocycloalkylene” refers to a heterocycloalkyl group having a valence of two; “heteroaryl” refers to an aromatic 4-8 membered monocyclic, 8-12 membered bicyclic, or 11-14 membered tricyclic ring system having 1-4 heteroatoms (if monocyclic), 1-6 heteroatoms (if bicyclic), or 1-9 heteroatoms (if tricyclic) that are each independently selected from N, O, S, Si, or P (e.g., carbon atoms and 1-3, 1-6, or 1-9 heteroatoms of N, O, or S, if monocyclic, bicyclic, or tricyclic, respectively). Examples of heteroaryl groups include pyridyl, furyl (furyl or furanyl), imidazolyl, benzimidazolyl, pyrimidinyl, thiophenyl or thienyl, quinolinyl, indolyl, thiazolyl, and the like; and “heteroarylene” refers to a heteroaryl group having a valence of two.

The term “halogen” means a monovalent substituent that is fluorine (fluoro), chlorine (chloro), bromine (bromo), or iodine (iodo). The prefix “halo” means a group including one or more of a fluoro, chloro, bromo, or iodo substituent instead of a hydrogen atom. A combination of halo groups (e.g., bromo and fluoro), or only fluoro groups may be present. For example, the term “haloalkyl” refers to an alkyl group substituted with one or more halogens. As used herein, “substituted C₁₋₈ haloalkyl” refers to a C₁₋₈ alkyl group substituted with at least one halogen, and is further substituted with one or more other substituent groups that are not halogens. It is to be understood that substitution of a group with a halogen atom is not to be considered a heteroatom-containing group, because a halogen atom does not replace a carbon atom.

Each of the foregoing substituent groups optionally may be substituted unless expressly provided otherwise. The term “optionally substituted” refers to being substituted or unsubstituted. “Substituted” means that at least one hydrogen atom of the chemical structure or group is replaced with another terminal substituent group that is typically monovalent, provided that the designated atom's normal valence is not exceeded. When the substituent is oxo (i.e., O), then two geminal hydrogen atoms on the carbon atom are replaced with the terminal oxo group. It is further noted that the oxo group is bonded to carbon via a double bond to form a carbonyl (C═O), where the carbonyl group is represented herein as —C(O)—. Combinations of substituents or variables are permissible. Exemplary substituent groups that may be present on a “substituted” position include, but are not limited to, nitro (—NO₂), cyano (—CN), hydroxyl (—OH), oxo (O), amino (—NH₂), mono- or di-(C₁₋₆)alkylamino, alkanoyl (such as a C₂₋₆ alkanoyl group such as acyl), formyl (—C(O)H), carboxylic acid or an alkali metal or ammonium salt thereof; esters (including acrylates, methacrylates, and lactones) such as C₂₋₆ alkyl esters (—C(O)O-alkyl or —OC(O)-alkyl) and C₇₋₁₃ aryl esters (—C(O)O-aryl or —OC(O)-aryl); amido (—C(O)NR₂ wherein R is hydrogen or C₁₋₆ alkyl), carboxamido (—CH₂C(O)NR₂ wherein R is hydrogen or C₁₋₆ alkyl), halogen, thiol (—SH), C₁₋₆ alkylthio (—S-alkyl), thiocyano (—SCN), C₁₋₆ alkyl, C₂₋₆ alkenyl, C₂₋₆ alkynyl, C₁₋₆ haloalkyl, C₁₋₉ alkoxy, C₁₋₆ haloalkoxy, C₃₋₁₂ cycloalkyl, C₅₋₁₈ cycloalkenyl, C₂₋₁₈ heterocycloalkenyl, C₆₋₁₂ aryl having at least one aromatic ring (e.g., phenyl, biphenyl, naphthyl, or the like, each ring either substituted or unsubstituted aromatic), C₇₋₁₉ arylalkyl having 1 to 3 separate or fused rings and from 6 to 18 ring carbon atoms, arylalkoxy having 1 to 3 separate or fused rings and from 6 to 18 ring carbon atoms, C₇₋₁₂ alkylaryl, C₃₋₁₂ heterocycloalkyl, C₃₋₁₂ heteroaryl, C₁₋₆ alkyl sulfonyl (—S(O)₂-alkyl), C₆₋₁₂ arylsulfonyl (—S(O)₂-aryl), or tosyl (CH₃C₆H₄SO₂—).

As used herein, when a definition is not otherwise provided, a “divalent linking group” refers to a divalent group including one or more of —O—, —S—, —Te—, —Se—, —C(O)—, —C(O)O—, —N(R′)—, —C(O)N(R′)—, —S(O)—, —S(O)₂—, —C(S)—, —C(Te)—, —C(Se)—, substituted or unsubstituted C₁₋₃₀ alkylene, substituted or unsubstituted C₃₋₃₀ cycloalkylene, substituted or unsubstituted C₃₋₃₀ heterocycloalkylene, substituted or unsubstituted C₆₋₃₀ arylene, substituted or unsubstituted C₃₋₃₀ heteroarylene, or a combination thereof, wherein each R′ is independently hydrogen, substituted or unsubstituted C₁₋₂₀ alkyl, substituted or unsubstituted C₁₋₂₀ heteroalkyl, substituted or unsubstituted C₆₋₃₀ aryl, or substituted or unsubstituted C₃₋₃₀ heteroaryl. Typically, the divalent linking group includes one or more of —O—, —S—, —C(O)—, —C(O)O—, —N(R′)—, —C(O)N(R′)—, —S(O)—, —S(O)₂—, substituted or unsubstituted C₁₋₃₀ alkylene, substituted or unsubstituted C₃₋₃₀ cycloalkylene, substituted or unsubstituted C₃₋₃₀ heterocycloalkylene, substituted or unsubstituted C₆₋₃₀ arylene, substituted or unsubstituted C₃₋₃₀ heteroarylene, or a combination thereof, wherein R′ is hydrogen, substituted or unsubstituted C₁₋₂₀ alkyl, substituted or unsubstituted C₁₋₂₀ heteroalkyl, substituted or unsubstituted C₆₋₃₀ aryl, or substituted or unsubstituted C₃₋₃₀ heteroaryl. More typically, the divalent linking group includes at least one of —O—, —C(O)—, —C(O)O—, —N(R′)—, —C(O)N(R′)—, substituted or unsubstituted C₁₋₁₀ alkylene, substituted or unsubstituted C₃₋₁₀ cycloalkylene, substituted or unsubstituted C₃₋₁₀ heterocycloalkylene, substituted or unsubstituted C₆₋₁₀ arylene, substituted or unsubstituted C₃₋₁₀ heteroarylene, or a combination thereof, wherein R is hydrogen, substituted or unsubstituted C₁₋₁₀ alkyl, substituted or unsubstituted C₁₋₁₀ heteroalkyl, substituted or unsubstituted C₆₋₁₀ aryl, or substituted or unsubstituted C₃₋₁₀ heteroaryl.

As used herein, an “acid-labile group” refers to a group in which a bond is cleaved by the action of an acid, optionally and typically with thermal treatment, resulting in formation of a polar group, such as a carboxylic acid or alcohol group. In some instances, the acid-labile group may be formed on a polymer, and optionally and typically with a moiety connected to the cleaved bond becoming disconnected from the polymer. In other systems, a non-polymeric compound may include an acid-labile group that may be cleaved by the action of an acid, resulting in formation of a polar group, such as a carboxylic acid or alcohol group on a cleaved portion of the non-polymeric compound. Such acid is typically a photo-generated acid with bond cleavage occurring during post-exposure baking (PEB); however, embodiments are not limited thereto, and, for example, such acid may be thermally generated. Suitable acid-labile groups include, for example: tertiary alkyl ester groups, secondary or tertiary ester groups having aryl groups, secondary or tertiary ester groups having a combination of alkyl and aryl groups, tertiary alkoxy groups, acetal groups, or ketal groups. Acid-labile groups are also commonly referred to in the art as “acid-cleavable groups,” “acid-cleavable protecting groups,” “acid-labile protecting groups,” “acid-leaving groups,” “acid-decomposable groups,” and “acid-sensitive groups.”

Sensitivity of photoresist performance is often correlated to the final device production throughput. In particular, high resolution lithographic techniques, such as 193 nm lithography (ArF), tend to struggle with photoresists having optimal sensitivity. To achieve good sensitivity, many photoresists employ photoacid generators (PAGs) containing an anion belonging to the class of sulfonate coupled with a polymer containing a low activation energy leaving group (e.g., an ester acetal or acetal-ester). In the last decade, many sulfonate derivatives have been developed for this purpose, with the example of fluorinated sulfonate being an example. This class of compounds, which have excelled in lithography thanks to their remarkably high acidity, are considered for replacement worldwide in favor of more sustainable alternatives. There remains a continuing need for PAG anions with good acidity and better sustainability.

The present inventors have discovered photoacid generators that include an anionic core that includes an aromatic group substituted with (i) an anion group, and (ii) an anion-stabilizing group that is configured to stabilize the anion group with an intramolecular non-covalent bond. In other words, the anion stabilizing group is configured to form an intramolecular non-covalent bond with the anion group. For example, without wishing to be bound to theory, the anion stabilizing group is capable of forming an intramolecular non-covalent bond with the anion group, or, for example, the anion-stabilizing group may form an intramolecular non-covalent bond with the anion group. In some embodiments, the intramolecular non-covalent bond may be formed in-situ, such as when the non-polymeric ionic photoacid generator compound is included in the photoresist composition. When used in photoresist compositions, PAGs in accordance with the invention can lead to suitable lithographic properties, such as for sizing energy (E_{size}), exposure latitude % (EL %), and/or linewidth roughness.

Provided is a polymerizable compound that includes an anion; a cation; and a polymerizable group bonded to the anion or the cation. The anion includes (i) a monocyclic or polycyclic C₃₋₆₀ aromatic group that includes a first substituent group, and (ii) an anion stabilizing group. The first substituent group includes an anion group, wherein the anion group is selected from a sulfonate anion group, a sulfinate anion group, a sulfonimide anion group, a sulfamate anion group, or a sulfonamide anion group. The anion stabilizing group is configured to form an intramolecular non-covalent bond with the anion group, wherein the first substituent group further includes the anion stabilizing group, or the monocyclic or polycyclic C₃₋₆₀ aromatic group further includes a second substituent group, wherein the second substituent group includes the anion stabilizing group. The anion stabilizing group may be a part of the first substituent group or the anion stabilizing group may be a part of a second substituent group of the monocyclic or polycyclic C₃₋₆₀ aromatic group.

As used herein, the “anion stabilizing group” refers to any suitable group that may stabilize the anion group via an intramolecular non-covalent bond, as provided herein. As such, the anion stabilizing group is configured to form an intramolecular non-covalent bond with the anion group, or, in other words, the anion stabilizing group is capable of forming an intramolecular non-covalent bond with the anion group. As used herein, the “non-covalent bond” may refer to any non-covalent bonding interaction between the anion stabilizing group and the anion group. As noted above, the non-covalent bonding interaction is intramolecular, where the anion stabilizing group and the anion group are on the same molecule. Exemplary non-covalent bonding includes hydrogen bonding or ionic bonding. The anion-stabilizing group may include a group that is protic. For example, the intramolecular non-covalent bond may be an intramolecular hydrogen bond between a suitable hydrogen atom of the anion stabilizing group and the anion group. For example, in some embodiments, the anion-stabilizing group may be configured to form an intramolecular hydrogen bond with the anion group, and, for example, in some embodiments, the anion-stabilizing group may form an intramolecular hydrogen bond with the anion group. In some embodiments, the intramolecular non-covalent bonding includes dipole-dipole interactions, ion-dipole interactions, or a combination thereof. As used herein, “non-covalent bond” does not include bonding based solely on Van der Waals forces.

The polymerizable group further enables the photoacid generator to be incorporated into a repeating unit of a polymer. The incorporation of the PAG into the polymer may further suppress acid diffusion during resist thermal processing, for example by physically constraining the PAG component to the remainder of the polymer. Thus, the diffusion of the PAG may be effectively limited, which reduces acid diffusion blur and may improve the resolution and pattern roughness. It is understood that suppressing acid diffusion may be manifested as increases to the contrast curve dose to clear or patterning dose-to-size. In addition, the bonding of the PAG moieties to the polymer chain may provide a more uniform spatial distribution of the PAG moieties throughout a resulting film, thus minimizing the randomness or stochastic effects that are typically encountered with small molecule PAGs in conventional chemical amplified resists.

In some aspects, the polymerizable group is bonded to the anion. For example, the anion may be represented by Formula (1):

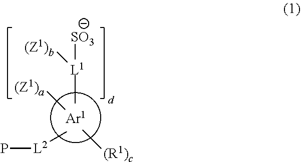

In Formula (1), Ar¹ is a monocyclic or polycyclic C₃₋₆₀ aromatic group. For example, the monocyclic or polycyclic C₃₋₆₀ aromatic group may be a monocyclic C₃₋₆₀ aromatic group or a polycyclic C₆₋₆₀ aromatic group. In an embodiment, the monocyclic or polycyclic C₃₋₆₀ aromatic group may be a monocyclic or polycyclic C₆₋₆₀ arylene group or a monocyclic or polycyclic C₃₋₆₀ heteroarylene group, typically a monocyclic or polycyclic C₆₋₃₀ arylene group or a monocyclic or polycyclic C₃₋₃₀ heteroarylene group.

It is to be understood that when the “monocyclic or polycyclic C₆₋₆₀ arylene group” is polycyclic, the number of carbon atoms is sufficient for the group to be chemically feasible. For example, the “monocyclic or polycyclic C₆₋₆₀ arylene group” may refer to “a monocyclic C₆ arylene group or a polycyclic C₁₀₋₆₀ arylene group”; or, for example “a monocyclic C₆ arylene group or a polycyclic C₁₀₋₃₀ arylene group”. Similarly, when the “monocyclic or polycyclic C₃₋₆₀ heteroarylene group” is polycyclic, the number of carbon atoms is sufficient for the group to be chemically feasible. For example, the “monocyclic or polycyclic C₃₋₆₀ heteroarylene group” may refer to “a monocyclic C₃₋₆ heteroarylene group or a polycyclic C₅₋₆₀ heteroarylene group”; or, for example “a monocyclic C₃₋₆ heteroarylene group or a polycyclic C₅₋₃₀ heteroarylene group”.

Exemplary monocyclic or polycyclic C₃₋₆₀ aromatic groups include, but are not limited to, benzene, naphthalene, anthracene, phenanthrene, pyrene, coronene, triphenylene, chrysene, phenalene, benz[a]anthracene, dibenz[a,h]anthracene, or benzo[a]pyrene.

In Formula (1), each L¹ is independently a single bond or one or more divalent linking groups, wherein L¹ is free of fluorine. In other words, when L¹ is one or more divalent linking groups, then L¹ is free of fluorine. Each of the one or more divalent linking groups may be substituted or unsubstituted. Exemplary divalent linking groups may each independently be selected from —O—, —C(O)—, —C(O)O—, —S—, —S(O)—, —S(O)₂—, —N(R′)—, —C(O)N(R′)—, substituted or unsubstituted C₁₋₃₀ alkylene, substituted or unsubstituted C₃₋₃₀ cycloalkylene, substituted or unsubstituted C₃₋₃₀ heterocycloalkylene, substituted or unsubstituted C₆₋₃₀ arylene, substituted or unsubstituted C₃₋₃₀ heteroarylene, or a combination thereof, wherein R′ may be hydrogen, substituted or unsubstituted C₁₋₂₀ alkyl, substituted or unsubstituted C₁₋₂₀ heteroalkyl, substituted or unsubstituted C₆₋₃₀ aryl, or substituted or unsubstituted C₃₋₃₀ heteroaryl. Typically, L¹ may be a single bond or substituted or unsubstituted C₁₋₂₀ alkylene, preferably a single bond or substituted or unsubstituted C₁₋₁₀ alkylene, wherein L¹ does not include an α-carbon atom covalently bonded directly to the sulfur atom of the sulfonate anion group that is substituted with a fluorine atom or a fluoroalkyl group.

It is to be understood that when b is 0, then L¹ may be a single bond or one or more linking groups, wherein L¹ is free of fluorine.

In Formula (1), each L² is independently a single bond or one or more divalent linking groups. Each of the one or more divalent linking groups may be substituted or unsubstituted. Exemplary divalent linking groups may each independently be selected from —O—, —C(O)—, —C(O)O—, —S—, —S(O)—, —S(O)₂—, —N(R′)—, —C(O)N(R′)—, substituted or unsubstituted C₁₋₃₀ alkylene, substituted or unsubstituted C₃₋₃₀ cycloalkylene, substituted or unsubstituted C₃₋₃₀ heterocycloalkylene, substituted or unsubstituted C₆₋₃₀ arylene, substituted or unsubstituted C₃₋₃₀ heteroarylene, or a combination thereof, wherein R′ may be hydrogen, substituted or unsubstituted C₁₋₂₀ alkyl, substituted or unsubstituted C₁₋₂₀ heteroalkyl, substituted or unsubstituted C₆₋₃₀ aryl, or substituted or unsubstituted C₃₋₃₀ heteroaryl. In some embodiments, L² comprises one or more divalent linking groups selected from —O—, —N(R′)—, —C(O)N(R′)—, —S(O)—, —S(O)₂—, substituted or unsubstituted C₁₋₃₀ alkylene, substituted or unsubstituted C₃₋₃₀ cycloalkylene, substituted or unsubstituted C₃₋₃₀ heterocycloalkylene, substituted or unsubstituted C₆₋₃₀ arylene, substituted or unsubstituted C₃₋₃₀ heteroarylene, or a combination thereof, wherein each R′ is independently hydrogen, substituted or unsubstituted C₁₋₂₀ alkyl, substituted or unsubstituted C₁₋₂₀ heteroalkyl, substituted or unsubstituted C₆₋₃₀ aryl, or substituted or unsubstituted C₃₋₃₀ heteroaryl. In some embodiments, L² includes an iodo-substituted aromatic group.

In Formula (1), each R¹ is independently a monovalent non-hydrogen substituent; wherein each R¹ optionally further comprises one or more divalent linking groups as part of its structure. For example, each R¹ may independently be halogen, hydroxyl, substituted or unsubstituted C₁₋₃₀ alkyl, substituted or unsubstituted C₃₋₃₀ cycloalkyl, substituted or unsubstituted C₃₋₃₀ cycloalkene, substituted or unsubstituted C₃₋₃₀ heterocycloalkyl, substituted or unsubstituted C₆₋₃₀ aryl, substituted or unsubstituted C₇₋₃₀ arylalkyl, substituted or unsubstituted C₇₋₃₀ alkylaryl, substituted or unsubstituted C₆₋₃₀ aryloxy, substituted or unsubstituted C₃₋₃₀ heteroaryl, substituted or unsubstituted C₄₋₃₀ alkylheteroaryl, substituted or unsubstituted C₄₋₃₀ heteroarylalkyl, or substituted or unsubstituted C₃₋₃₀ heteroaryloxy. Typically, each R¹ may independently be hydroxyl, substituted or unsubstituted C₁₋₃₀ alkyl, substituted or unsubstituted C₃₋₃₀ cycloalkyl, substituted or unsubstituted C₃₋₃₀ cycloalkene, substituted or unsubstituted C₃₋₃₀ heterocycloalkyl, substituted or unsubstituted C₆₋₃₀ aryl, substituted or unsubstituted C₇₋₃₀ arylalkyl, substituted or unsubstituted C₇₋₃₀ alkylaryl, substituted or unsubstituted C₆₋₃₀ aryloxy, substituted or unsubstituted C₃₋₃₀ heteroaryl, substituted or unsubstituted C₄₋₃₀ alkylheteroaryl, substituted or unsubstituted C₄₋₃₀ heteroarylalkyl, or substituted or unsubstituted C₃₋₃₀ heteroaryloxy. In some embodiments, at least one R¹ is a substituted C₆₋₃₀ aryl or a substituted C₇₋₃₀ arylalkyl. In some embodiments, at least one R¹ includes a halogen atom, for example a fluoro or an iodo group, as part of its structure.

In Formula (1), each R¹ optionally further comprises one or more divalent linking groups as part of its structure. Each of the one or more divalent linking groups may be substituted or unsubstituted. Exemplary divalent linking groups may be selected from —O—, —C(O)—, —C(O)O—, —S—, —S(O)—, —S(O)₂—, —N(R′)—, —C(O)N(R′)—, substituted or unsubstituted C₁₋₃₀ alkylene, substituted or unsubstituted C₃₋₃₀ cycloalkylene, substituted or unsubstituted C₃₋₃₀ heterocycloalkylene, substituted or unsubstituted C₆₋₃₀ arylene, substituted or unsubstituted C₃₋₃₀ heteroarylene, or a combination thereof, wherein R′ may be hydrogen, substituted or unsubstituted C₁₋₂₀ alkyl, substituted or unsubstituted C₁₋₂₀ heteroalkyl, substituted or unsubstituted C₆₋₃₀ aryl, or substituted or unsubstituted C₃₋₃₀ heteroaryl.

In some embodiments, one or more R¹ may each independently include an acid-labile group, a lactone-containing group, a base-solubilizing group, or the like, or a combination thereof.

In Formula (1), each Z¹ independently comprises an anion stabilizing group, wherein at least one Z¹ is configured to form an intramolecular non-covalent bond with the sulfonate anion group to form a ring having from 5 to 8 atoms, wherein Z¹ is independently chosen from —OH, —C(O)OH, —SH, —C(O)SH, —NHS(O)₂R², —S(O)₂R², —S(O)R², —S(O)₂NHS(O)₂R², —CH(═NOH), or —B(R³)₂; wherein each Z¹ optionally further comprises one or more divalent linking groups as part of its structure. Typically, each anion stabilizing group Z¹ may be independently chosen from —OH, C(O)OH, SH, or —B(OH)₂, and preferably at least one anion stabilizing group Z¹ comprises —OH.

In some embodiments, the anion-stabilizing group may have a pKa of 25 or less, typically 20 or less, or 18 or less, and preferably 16 or less.

In some embodiments, each anion stabilizing group comprises a group that is protic. For example, when the anion stabilizing group is protic, then each Z¹ may be independently chosen from —OH, —C(O)OH, —SH, —C(O)SH, —NHS(O)₂R², —S(O)₂R^{2a}, —S(O)₂NHS(O)₂R², —CH(═NOH), or —B(R^{2a})₂, wherein each R² is independently chosen from fluorine, hydroxyl, substituted or unsubstituted C₁₋₂₀ alkyl, substituted or unsubstituted C₁₋₂₀ heteroalkyl, substituted or unsubstituted C₆₋₃₀ aryl, or substituted or unsubstituted C₃₋₃₀ heteroaryl; each R^{2a} is hydroxyl; and each R^{3a} is independently chosen from hydrogen, fluorine, hydroxyl, substituted or unsubstituted C₁₋₂₀ alkyl, substituted or unsubstituted C₁₋₂₀ heteroalkyl, substituted or unsubstituted C₆₋₃₀ aryl, or substituted or unsubstituted C₃₋₃₀ heteroaryl, provided that at least one R^{3a} is hydrogen or hydroxyl.

In Formula (1), each Z¹ optionally further comprises one or more divalent linking groups as part of its structure. Each of the one or more divalent linking groups may be substituted or unsubstituted. Exemplary divalent linking groups may be selected from —O—, —C(O)—, —C(O)O—, —S—, —S(O)—, —S(O)₂—, —N(R′)—, —C(O)N(R′)—, substituted or unsubstituted C₁₋₃₀ alkylene, substituted or unsubstituted C₃₋₃₀ cycloalkylene, substituted or unsubstituted C₃₋₃₀ heterocycloalkylene, substituted or unsubstituted C₆₋₃₀ arylene, substituted or unsubstituted C₃₋₃₀ heteroarylene, or a combination thereof, wherein R′ may be hydrogen, substituted or unsubstituted C₁₋₂₀ alkyl, substituted or unsubstituted C₁₋₂₀ heteroalkyl, substituted or unsubstituted C₆₋₃₀ aryl, or substituted or unsubstituted C₃₋₃₀ heteroaryl. Typically, Z¹ may optionally further include one or more divalent linking groups selected from —O—, —C(O)—, —C(O)O—, —S(O)—, —S(O)₂—, —N(R′)—, —C(O)N(R′)—, substituted or unsubstituted C₁₋₁₀ alkylene, substituted or unsubstituted C₃₋₁₀ cycloalkylene, substituted or unsubstituted C₃₋₁₀ heterocycloalkylene, substituted or unsubstituted C₆₋₃₀ arylene, substituted or unsubstituted C₃₋₃₀ heteroarylene, or a combination thereof, wherein R′ may be hydrogen, substituted or unsubstituted C₁₋₂₀ alkyl, substituted or unsubstituted C₁₋₂₀ heteroalkyl, substituted or unsubstituted C₆₋₃₀ aryl, or substituted or unsubstituted C₃₋₃₀ heteroaryl.

In Formula (1), each R² is independently chosen from fluorine, trifluoromethyl, hydroxyl, substituted or unsubstituted C₁₋₂₀ alkyl, substituted or unsubstituted C₁₋₂₀ heteroalkyl, substituted or unsubstituted C₆₋₃₀ aryl, or substituted or unsubstituted C₃₋₃₀ heteroaryl.

In Formula (1), each R^{2a} is independently chosen from hydrogen, fluorine, hydroxyl, substituted or unsubstituted C₁₋₂₀ alkyl, substituted or unsubstituted C₁₋₂₀ heteroalkyl, substituted or unsubstituted C₆₋₃₀ aryl, or substituted or unsubstituted C₃₋₃₀ heteroaryl.

In Formula (1), two R¹ together optionally form a fused ring with Ar¹, wherein the fused ring optionally further comprises one or more divalent linking groups as part of its structure. Each of the one or more divalent linking groups is substituted or unsubstituted, and the fused ring is substituted or unsubstituted.

In Formula (1), one Z¹ and one R¹ together optionally form a fused ring with Ar¹, wherein the fused ring optionally further comprises one or more divalent linking groups as part of its structure. Each of the one or more divalent linking groups is substituted or unsubstituted, and wherein the fused ring is substituted or unsubstituted. The fused ring that is formed with Ar¹ may be aliphatic or aromatic.

In Formula (1), P is a polymerizable group. Exemplary polymerizable groups include, but are not limited to, a (meth)acrylate group, a vinyl group, a vinyl aromatic group, a vinyl ether group, a vinyl ketone group, a vinyl ester group, an epoxy group, or the like, or a combination thereof.

In Formula (1), each a and b is independently an integer from 0 to 2, provided that a sum of a and b is 1 or greater. Typically, a is 1 or 2, and b is 0.

In Formula (1), c is an integer from 0 to 10. Typically, c is an integer from 0 to 2, preferably c is 0 or 1.

In Formula (1), d is an integer from 1 to 3. Typically, d is 1 or 2, preferably d is 1. For example, in some embodiments, a may be 1 or 2 and d may be 1. In some embodiments, d may be 1 and L¹ is a single bond. It is to be understood that when L¹ is a single bond, then b is 0.

In some embodiments, Ar¹ may be a monocyclic C₃₋₆₀ aromatic group and at least one Z¹ is located at an ortho position to a group represented by -L¹-SO₃⁻. For example, Ar¹ may be a monocyclic C₃₋₆ aromatic group and at least one Z₁ may be located at an ortho position to the group represented by -L¹-SO₃⁻. In some embodiments, Ar¹ may be a monocyclic C₃₋₆ aromatic group, L¹ is a single bond, and at least one Z₁ may be located at an ortho position to the group represented by -L¹-SO₃⁻.

In some embodiments, Ar¹ may be a polycyclic C₆₋₆₀ aromatic group and at least one Z¹ group may be bonded to a ring carbon atom of Ar¹ located at an ortho position on a same ring to the group represented by -L¹-SO₃⁻. For example, Ar¹ may be a polycyclic C₆₋₆₀ aromatic group, L¹ is a single bond, and at least one Z¹ group may be bonded to a ring carbon atom of Ar¹ located at an ortho position on a same ring to the group represented by -L¹-SO₃⁻.

In some embodiments, in Formula (1), Ar¹ may be a polycyclic C₆₋₆₀ aromatic group and at least one Z¹ group may be bonded to a ring carbon atom of Ar¹ that is at a beta position with respect to a ring carbon atom to which -L¹-SO₃⁻ is bonded, and the at least one Z¹ group and -L¹-SO₃⁻ are bonded to different rings of the polycyclic aromatic group. As used herein, the term “substituent groups at a beta position” refers to substituent groups that are bonded to a respective aromatic ring carbon atom on different rings of the polycyclic ring system, which ring carbon atoms are separated by a joined ring carbon atom.

In some embodiments, the anion may be represented by Formula (1a):

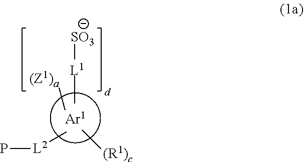

wherein Ar¹, R¹, Z¹, L¹, L², P, a, c, and d are each as defined for Formula (1).

In some embodiments, the anion is of Formula (1b):

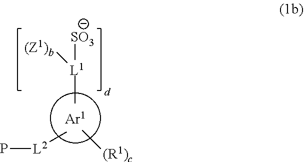

wherein Ar¹, R¹, Z¹, L¹, L², P, b, c, and d are each as defined for Formula (1).

Exemplary anions represented by Formula (1) include the following:

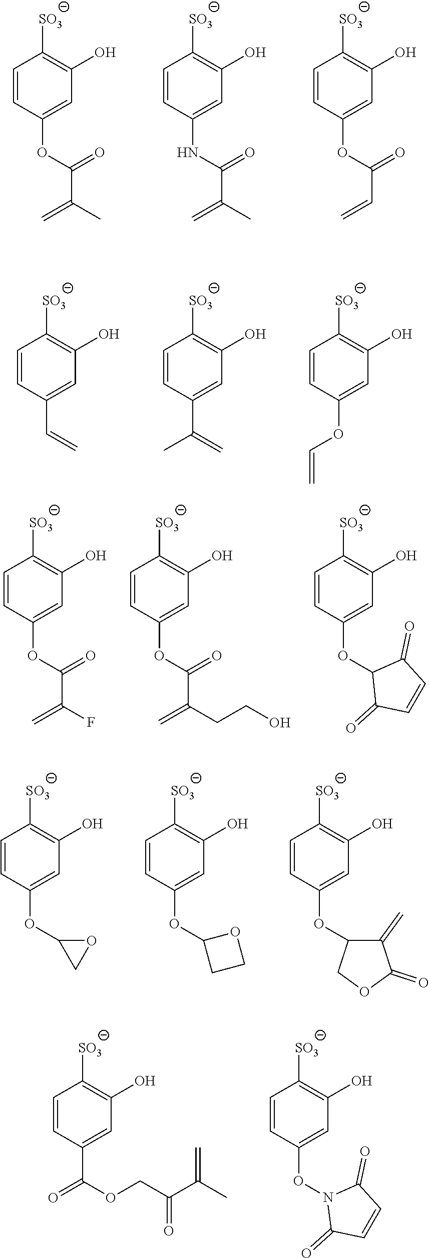

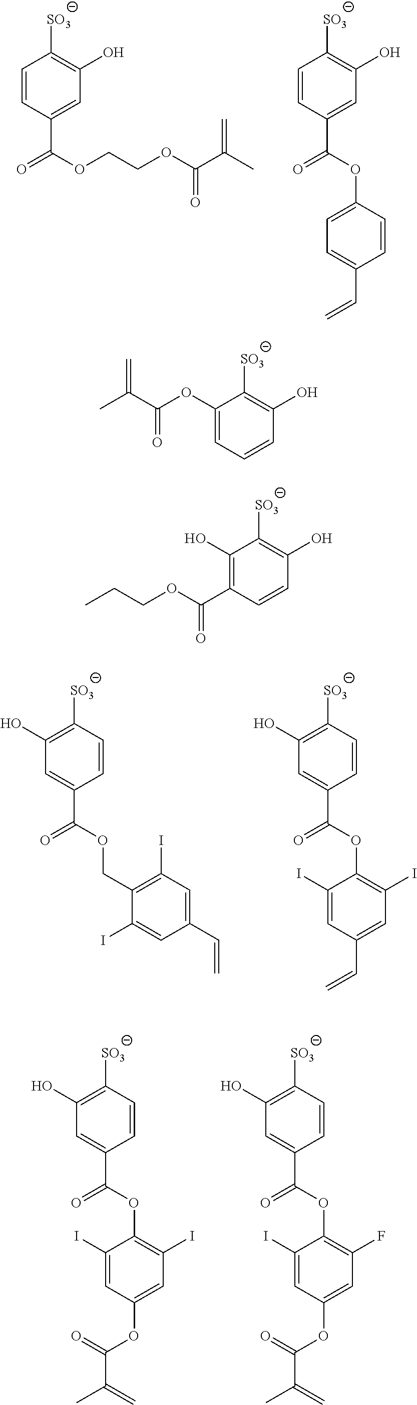

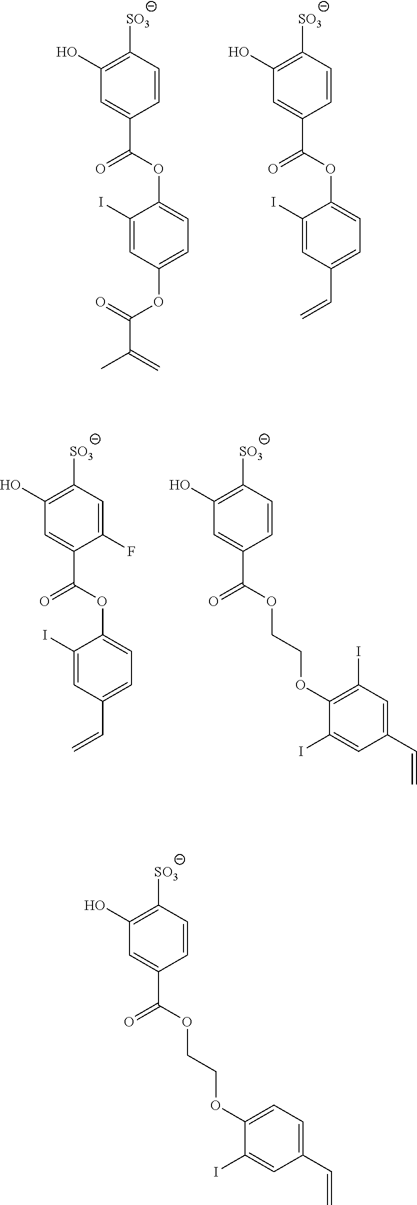

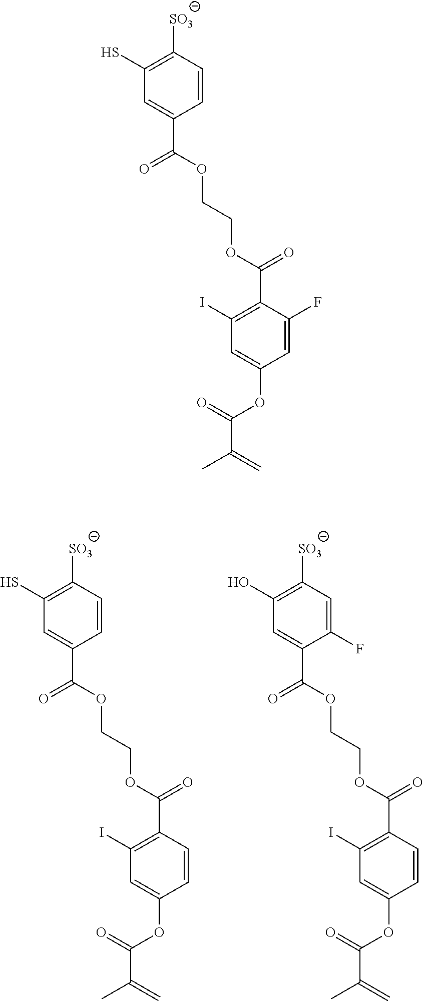

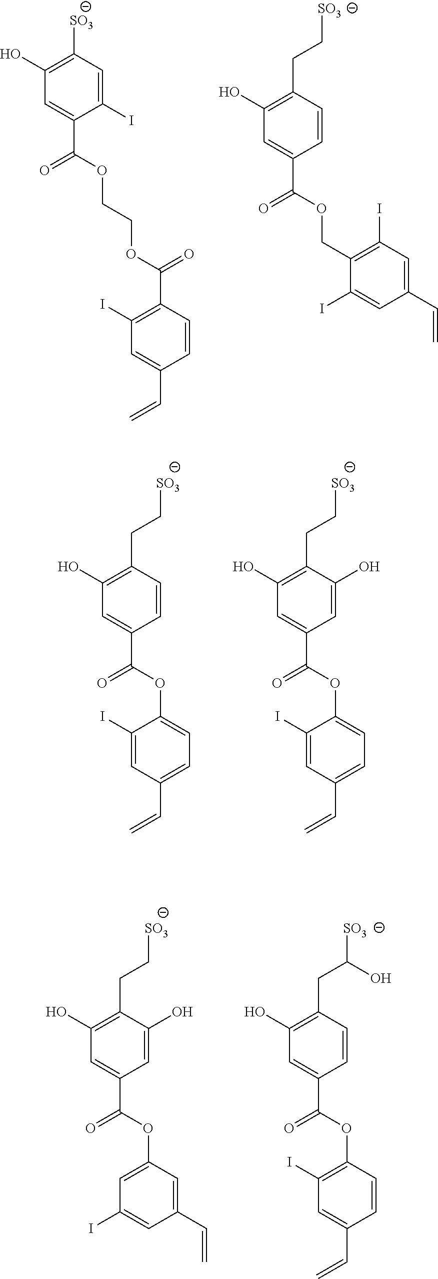

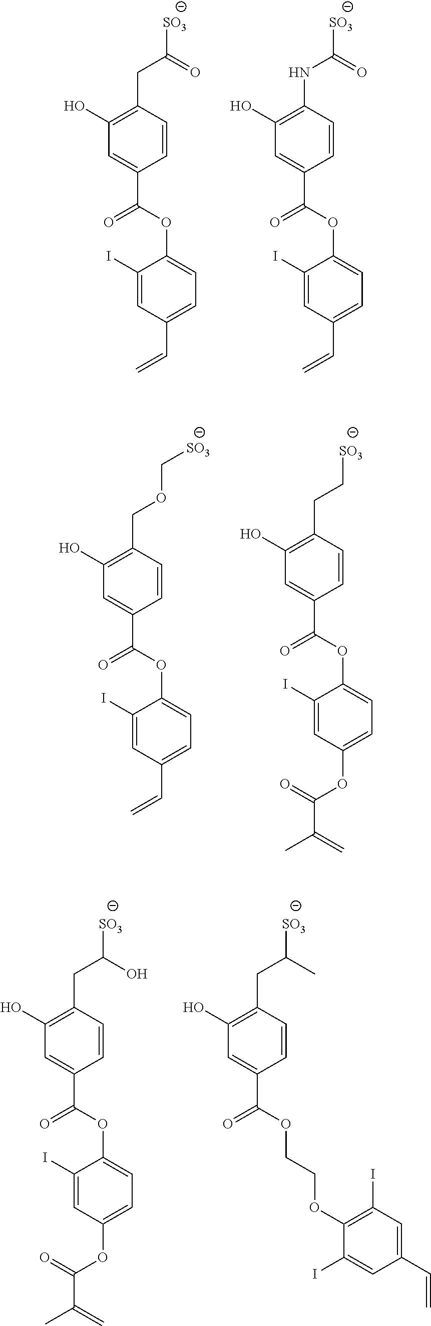

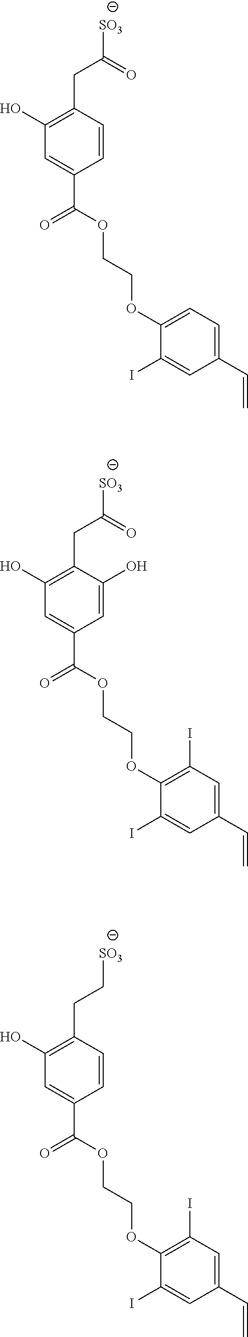

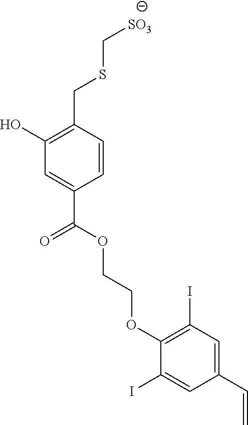

In some embodiments, the anion may be free of trifluoromethyl groups and difluoromethylene groups. In other words, in some embodiments, the anion of Formula (1) may be free of trifluoromethyl groups and difluoromethylene groups. For example, in some embodiments, the anion does not include fluorine (the anion of Formula (1) may be free of fluorine).

In some aspects, the polymerizable group is bonded to the cation of the polymerizable compound. For example, the anion may be represented by Formula (2):

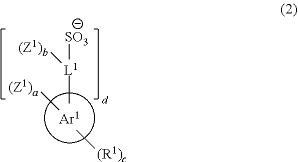

wherein Ar¹, R¹, Z¹, Z², L¹, L², P, a, b, c, and d are each as defined for Formula (1).

Exemplary anions represented by Formula (2) include the following:

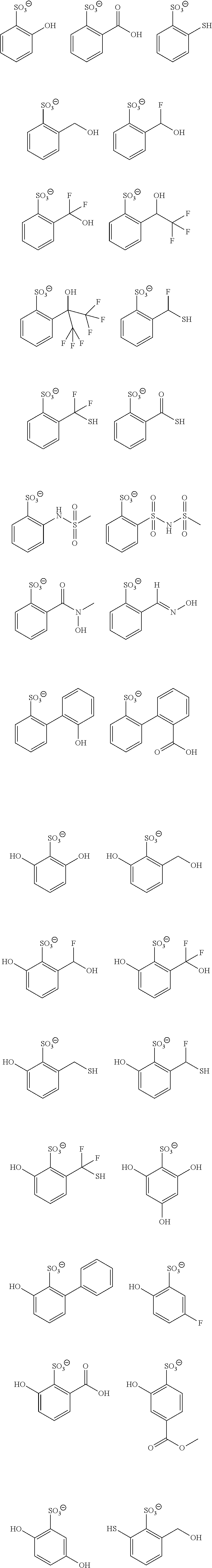

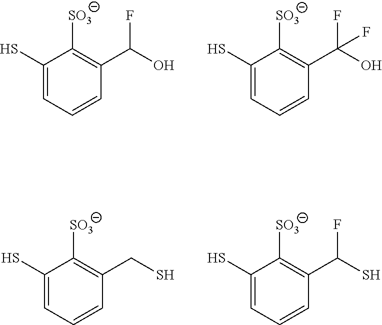

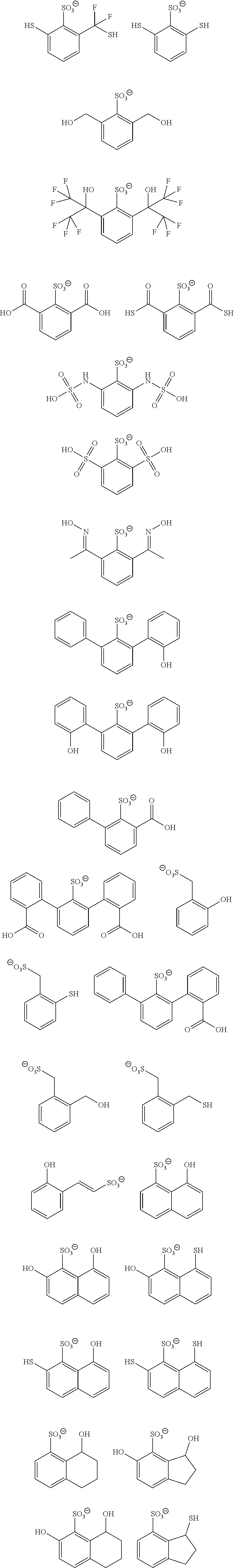

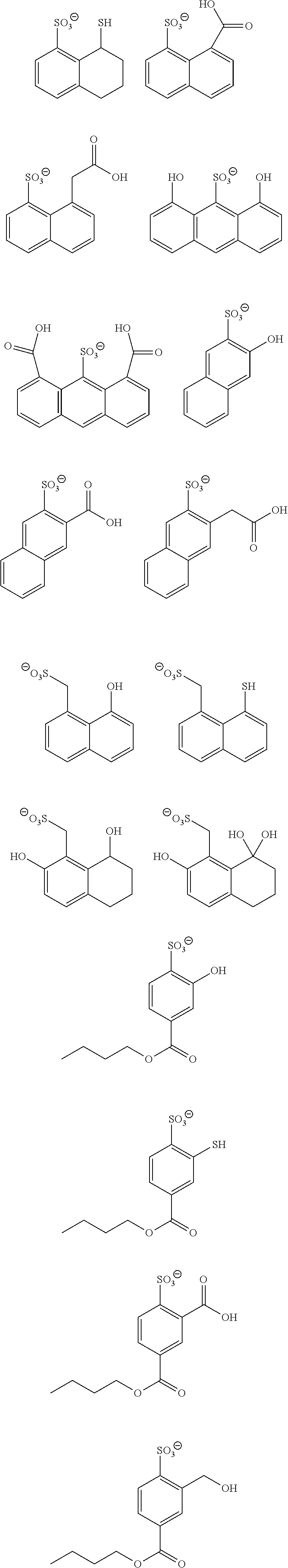

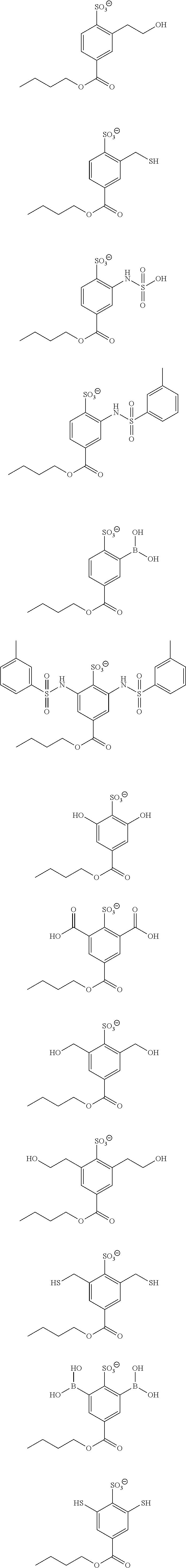

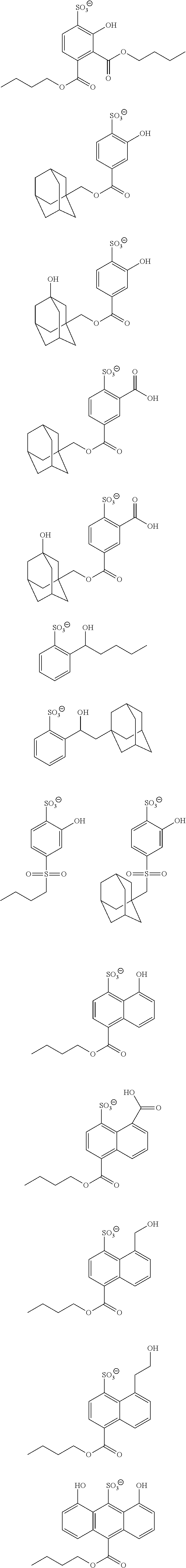

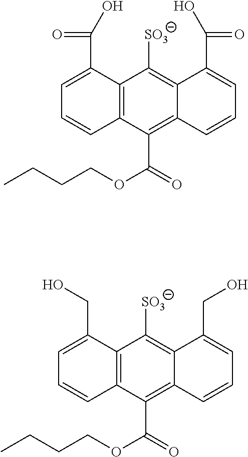

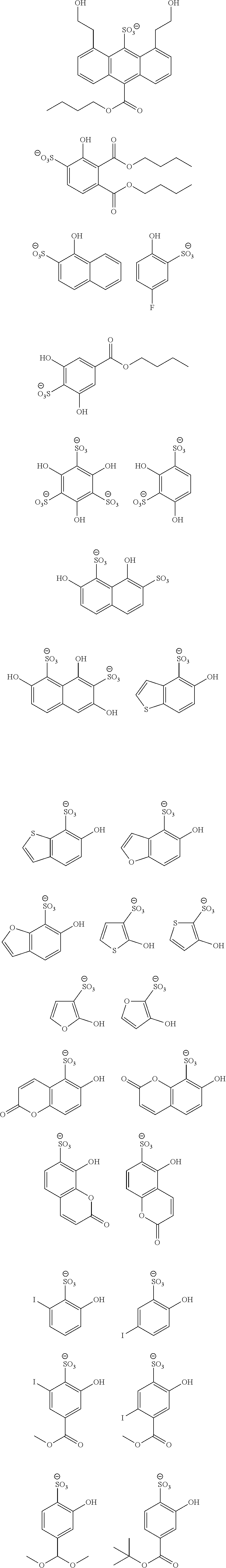

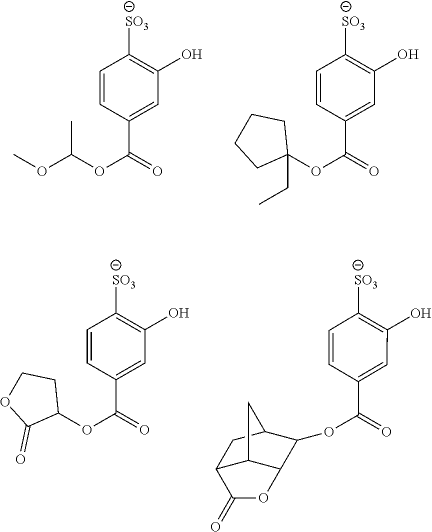

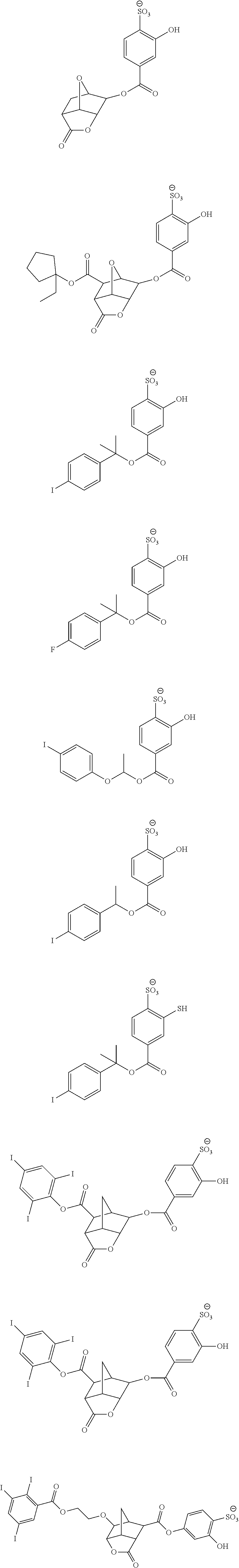

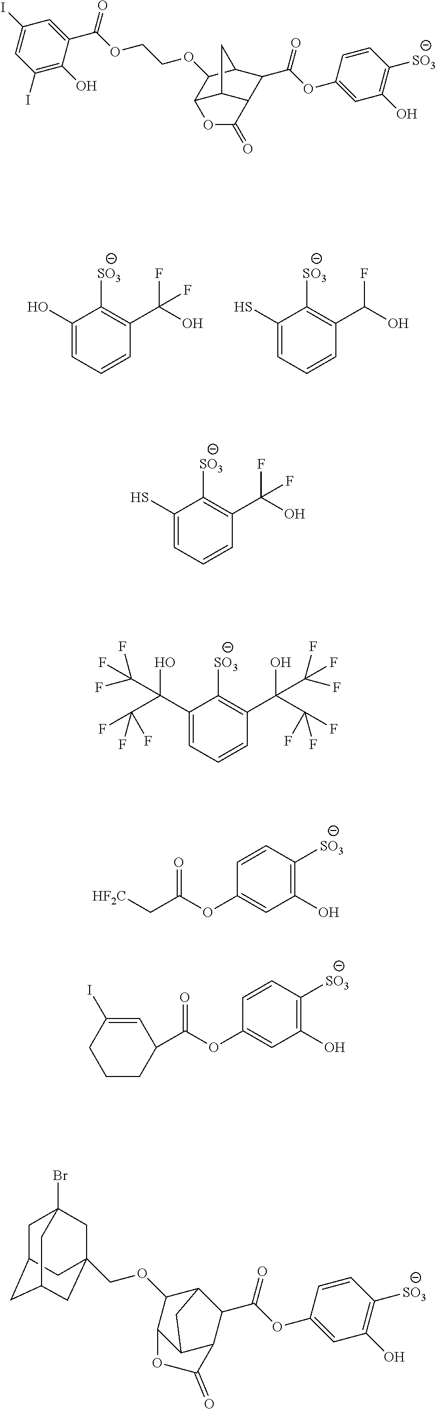

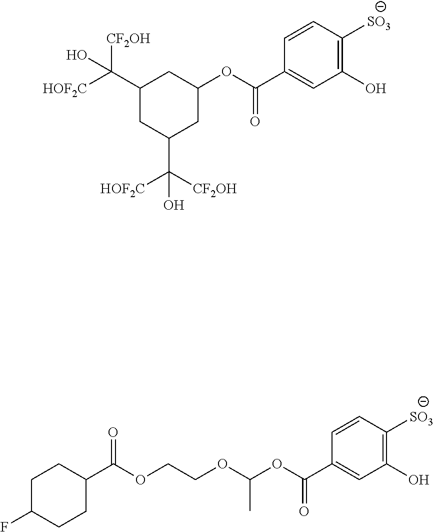

In some embodiments, a conjugate acid of the anion of the polymerizable compound may have a pKa of 0 or less. Typically, the conjugate acid may have a pKa of −2 or less, preferably −5 or less. The conjugate acid may, for example, have a pKa from −15 to 0 or from −15 to −2.

The anions of the photoacid generator compound may be obtained from commercial sources or prepared by any suitable method. For example, such anions may be prepared as described in the Examples herein.

The polymerizable compound further includes a cation. It is to be understood that when the anion portion does not include a polymerizable group, then the cation portion will include a polymerizable group as a part of its structure. In some embodiments, both the cation and the anion each include a polymerizable group as part of their structures.

Any suitable cation may be used. Exemplary cations may include ammonium, pyridinium, iodonium, and sulfonium, but embodiments are not limited thereto.

In some embodiments, the cation may be a sulfonium cation of Formula (3a) or an iodonium cation of Formula (3b):

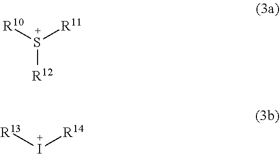

In Formulae (3a) and (3b), R¹⁰ to R¹⁴ are each independently substituted or unsubstituted C₁₋₃₀ alkyl, substituted or unsubstituted C₃₋₃₀ cycloalkyl, substituted or unsubstituted C₂₋₃₀ alkenyl, substituted or unsubstituted C₂₋₃₀ alkynyl, substituted or unsubstituted C₆₋₃₀ aryl, substituted or unsubstituted C₃₋₃₀ heteroaryl, substituted or unsubstituted C₇₋₃₀ arylalkyl, or substituted or unsubstituted C₄₋₃₀ heteroarylalkyl, or combinations thereof. Each of R¹⁰ to R¹⁴ may be either separate or connected to another group R¹⁰ to R¹⁴ via a single bond or a divalent linking group to form a ring. Each of R¹⁰ to R¹⁴ optionally may include as part of its structure a divalent linking group. Each of R¹⁰ to R¹⁴ independently may optionally comprise an acid-labile group chosen, for example, from tertiary alkyl ester groups, secondary or tertiary aryl ester groups, secondary or tertiary ester groups having a combination of alkyl and aryl groups, tertiary alkoxy groups, acetal groups, or ketal groups. When the anion does not include a polymerizable group, at least one of R¹⁰ to R¹² or at least one of R¹³ to R¹⁴ further includes a polymerizable group as part of its structure. Exemplary polymerizable groups include, but are not limited to, a (meth)acrylate group, an epoxy group, a vinyl aromatic group, a vinyl ether group, a vinyl ketone group, and/or a vinyl ester group.

Exemplary sulfonium cations of Formula (3a) may include one or more of the following:

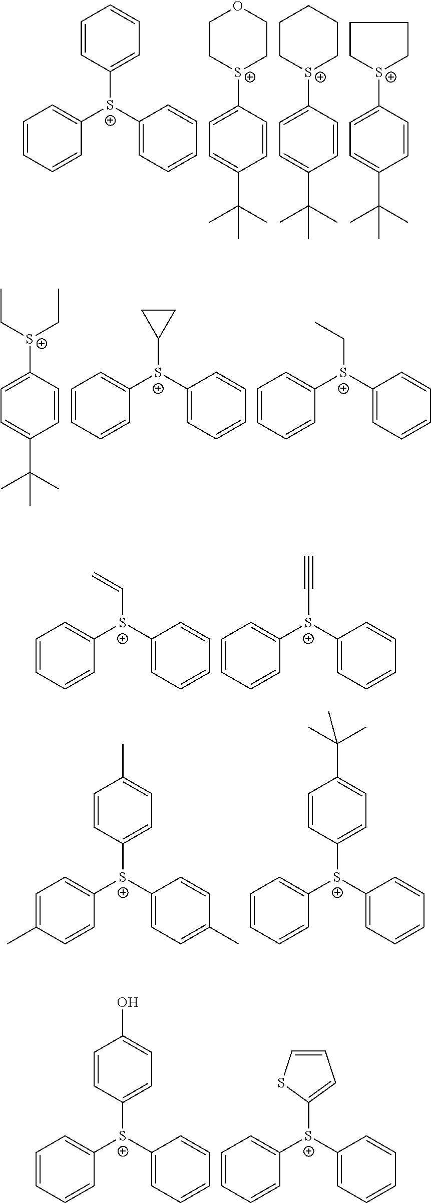

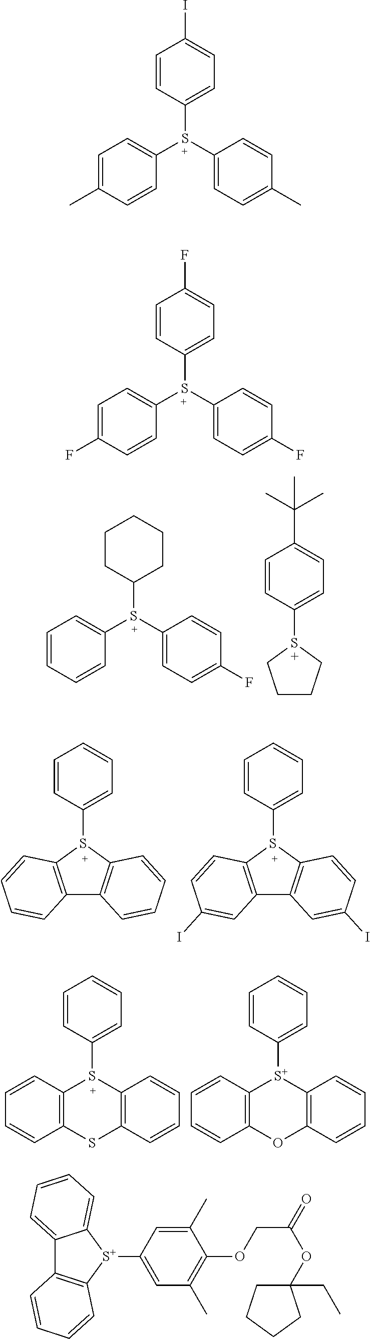

Exemplary iodonium cations of formula (3b) may include one or more of the following:

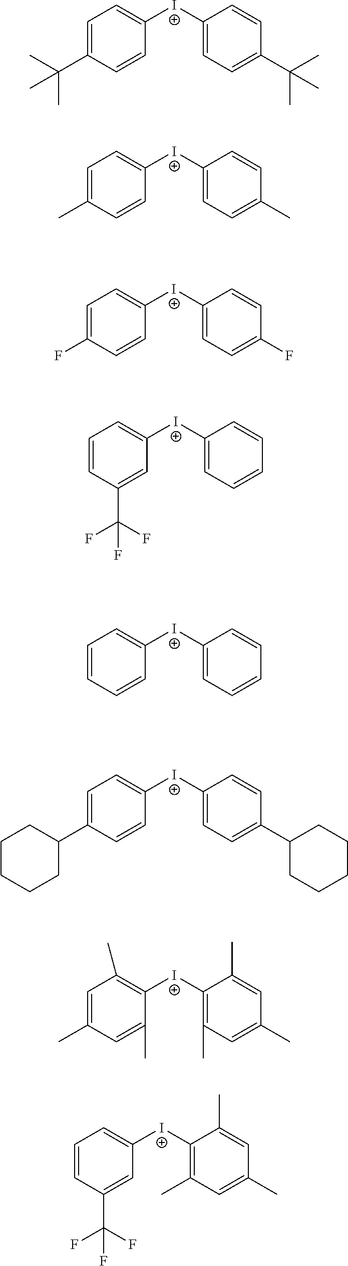

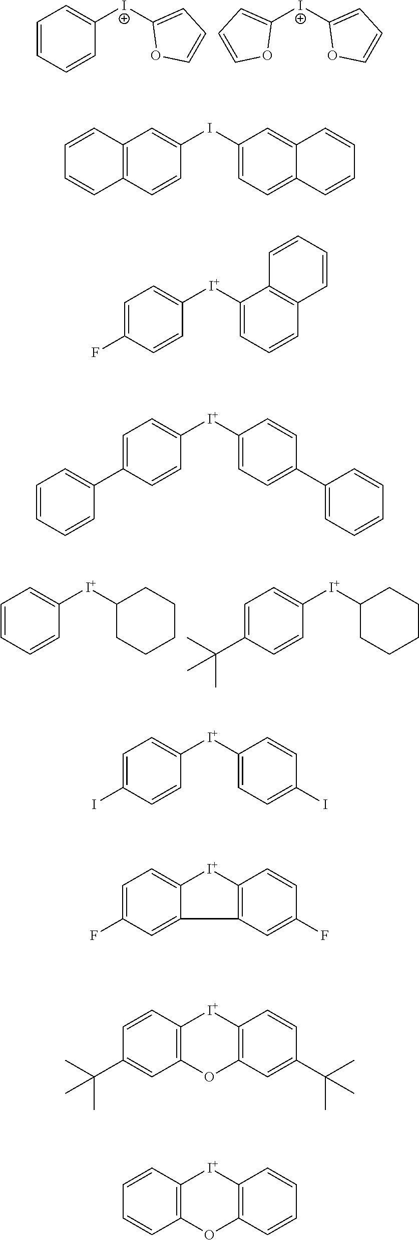

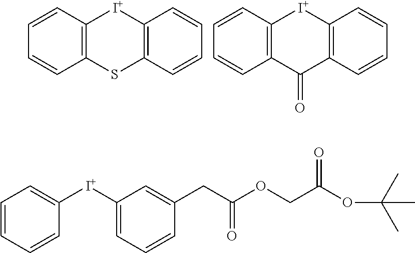 [SMILES: c1ccc2c(c1)Sc1ccccc1[I+]2 ; O=C1c2ccccc2[I+]c2ccccc21 ; CC(C)(C)OC(=O)COC(=O)Cc1cccc([I+]c2ccccc2)c1]

Exemplary cations that include a polymerizable group include the following:

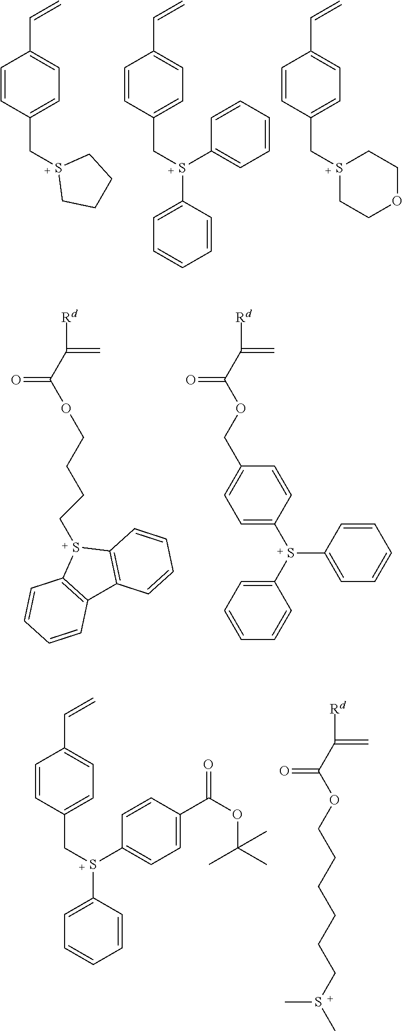

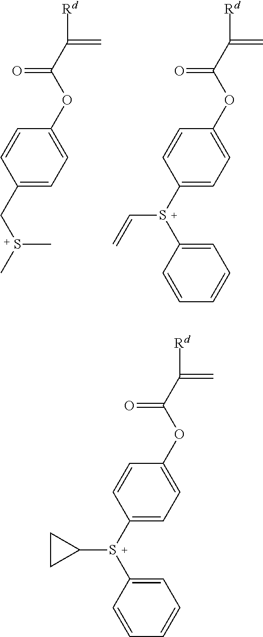

wherein each R^{d} is independently hydrogen, deuterium, fluorine, cyano, or substituted or unsubstituted C₁₋₁₀ alkyl. Preferably, R^{d} is hydrogen, fluorine, or substituted or unsubstituted C₁₋₅ alkyl, typically methyl.

The cations for the polymerizable compounds may be obtained from commercial sources or prepared using common synthetic procedures.

Suitable polymerizable compounds include those resulting from any combination of the above-described anions and cations. The polymerizable compounds may be prepared by combining the anion and the cation species under appropriate conditions.

Another aspect provides a polymer that includes a repeating unit derived from the polymerizable compound. Other repeating units may be included in the polymer, as described herein for the repeating units of the non-solvent alkali-insoluble base material polymers.

Also provided is a photoresist composition that includes the polymerizable compound, or a polymer comprising a repeating unit derived from the polymerizable compound, and a solvent. That is, the photoresist composition may include (i) the polymerizable compound, and a solvent, or (ii) the photoresist composition may include a polymer including a repeating unit that is derived from the polymerizable compound, and a solvent.

The polymerizable compound may be included in the photoresist composition in an amount from 1 to 99 weight percent (wt %), more typically from 1 to 80%, 2 to 75 wt %, or from 2 to 60 wt %, based on total solids of the photoresist composition.

When the photoresist composition includes a polymer including a repeating unit that is derived from the polymerizable compound, the polymer may be included in the photoresist composition in an amount from 1 to 99 weight percent (wt %), more typically from 1 to 80%, 2 to 75 wt %, or from 2 to 60 wt %, based on total solids of the photoresist composition.

The repeating unit of the polymer including a unit derived from the polymerizable compound typically may be present in an amount from 1 to 100 mol %, typically from 1 to 55 mol %, more typically from 2 to 25 mol %, based on total repeating units of the polymer. Other repeating units may be included in the polymer, as described herein for the repeating units of the non-solvent alkali-insoluble base material polymers.

Exemplary polymers including a repeating unit derived from the polymerizable compound include the following:

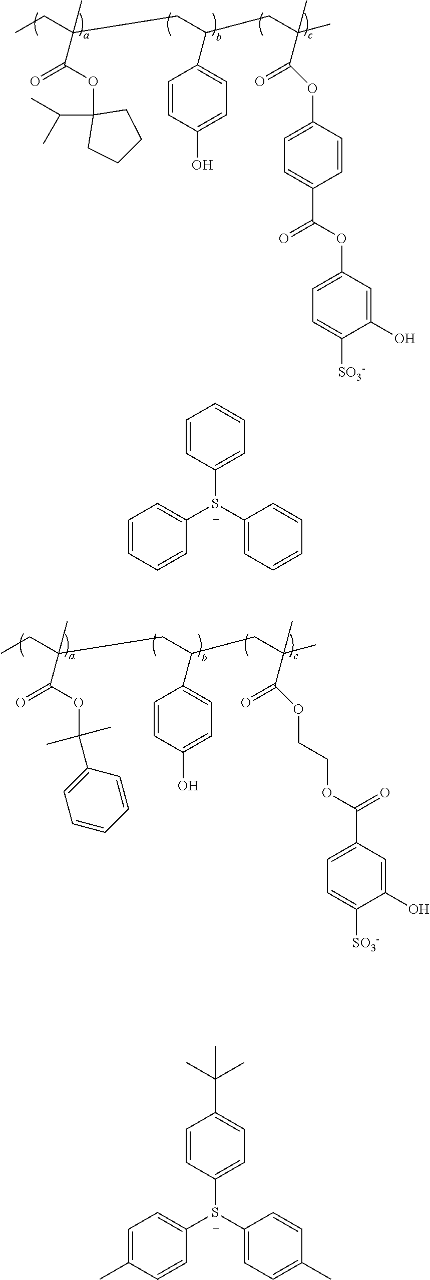

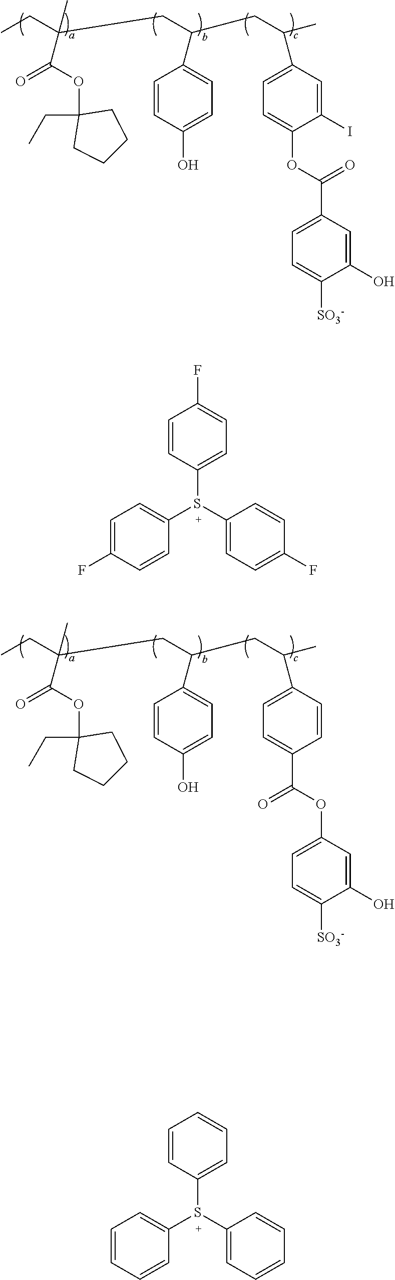

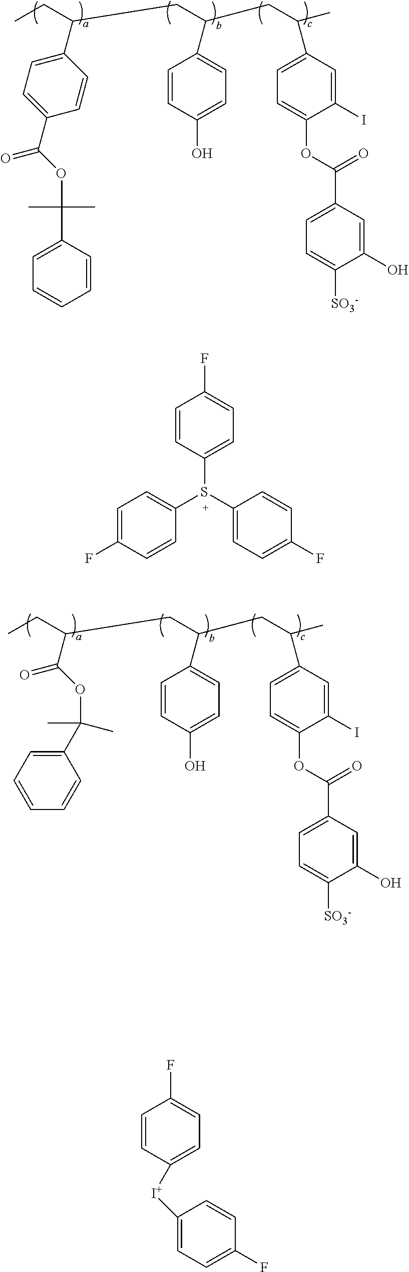

wherein a, b, and c represent the mole fractions for the respective repeating units of the polymer and a+b+c=1.

The polymer typically has a weight average molecular weight (M_{w}) from 1,000 to 200,000 Dalton (Da), preferably from 10,000 to 150,000 Da, more preferably 15,000 to 150,000 Da, and still more preferably from 25,000 to 150,000 Da or from 50,000 to 150,000 Da. The polydispersity index (PDI) of the polymer, which is the ratio of M_{w} to number average molecular weight (Mₙ) is typically from 1.1 to 3, and more typically from 1.1 to 2. Molecular weight values are determined by gel permeation chromatography (GPC) using polystyrene standards.

The polymer may be prepared using any suitable method(s) in the art. For example, one or more monomers corresponding to the repeating units described herein may be combined, or fed separately, using suitable solvent(s) and initiator, and polymerized in a reactor. For example, the polymers may be obtained by polymerization of the respective monomers under any suitable conditions, such as by heating at an effective temperature, irradiation with actinic radiation at an effective wavelength, or a combination thereof.

The photoresist composition may further include an additional photoacid generator that is different from the polymerizable compound. The additional PAG may be in polymeric or non-polymeric form. In polymeric form, the additional PAG may be present as a moiety in a repeating unit of a polymer that is derived from a polymerizable PAG monomer.

Suitable additional PAG compounds maybe of the formula G⁺A⁻, wherein G⁺ is a photoactive cation and A⁻ is an anion that can generate a photoacid. The photoactive cation is preferably chosen from onium cations, preferably iodonium or sulfonium cations such as those described above with respect to the inventive non-polymeric ionic photoacid generator compounds (e.g., those of Formulae (3a) and/or (3b)). Particularly suitable anions include those whose conjugated acids have a pKa of from −15 to 0, or from −14 to 0, or from −13 to 0. The anion is typically an organic anion having a sulfonate group or a non-sulfonate-type group, such as sulfonamidate, sulfonimidate, methide, arsenate, or borate. In some embodiments, the additional PAG may have an anion having a structure of Formula (1) as defined for the anion of the non-polymeric ionic photoacid generator compound, wherein the anion of the additional PAG compound does not include a group Z¹ that is an anion stabilizing group.

In some aspects, the anion of the additional PAG does not include and is free of —F, —CF₃, or —CF₂— groups. It should be understood that “free of —F, —CF₃, or —CF₂— groups” means that the anion of the additional PAG excludes groups such as —CH₂CF₃ and —CH₂CF₂CH₃. In still other aspects, the anion of the additional PAG is free of fluorine (i.e., does not contain a fluorine atom and is not substituted by a fluorine-containing group). In some aspects, the additional PAG is free of fluorine (i.e., both the photoactive cation and the anion are free of fluorine).

Exemplary onium salts may include, for example, triphenylsulfonium trifluoromethanesulfonate, (p-tert-butoxyphenyl)diphenylsulfonium trifluoromethanesulfonate, tris(p-tert-butoxyphenyl)sulfonium trifluoromethanesulfonate, triphenylsulfonium p-toluenesulfonate; di-t-butyphenyliodonium perfluorobutanesulfonate, and di-t-butyphenyliodonium camphorsulfonate. Other useful additional PAG compounds are known in the art of chemically amplified photoresists and include, for example: non-ionic sulfonyl compounds, for example, 2-nitrobenzyl-p-toluenesulfonate, 2,6-dinitrobenzyl-p-toluenesulfonate, and 2,4-dinitrobenzyl-p-toluenesulfonate; sulfonic acid esters, for example, 1,2,3-tris(methanesulfonyloxy)benzene, 1,2,3-tris(trifluoromethanesulfonyloxy)benzene, and 1,2,3-tris(p-toluenesulfonyloxy)benzene; diazomethane derivatives, for example, bis(benzenesulfonyl)diazomethane, bis(p-toluenesulfonyl)diazomethane; glyoxime derivatives, for example, bis-O-(p-toluenesulfonyl)-α-dimethylglyoxime, and bis-O-(n-butanesulfonyl)-α-dimethylglyoxime; sulfonic acid ester derivatives of an N-hydroxyimide compound, for example, N-hydroxysuccinimide methanesulfonic acid ester, N-hydroxysuccinimide trifluoromethanesulfonic acid ester; and halogen-containing triazine compounds, for example, 2-(4-methoxyphenyl)-4,6-bis(trichloromethyl)-1,3,5-triazine, and 2-(4-methoxynaphthyl)-4,6-bis(trichloromethyl)-1,3,5-triazine. Suitable additional PAGs are further described in U.S. Pat. Nos. 8,431,325 and 4,189,323.

Typically, when the photoresist composition includes an additional PAG, the additional PAG is present in the photoresist composition in an amount of from 0.1 to 55 wt %, more typically 1 to 25 wt %, based on total solids of the photoresist composition. When present in polymeric form, the additional PAG is typically included in a polymer in an amount from 1 to 25 mol %, more typically from 1 to 8 mol %, or from 2 to 6 mol %, based on total repeating units in the polymer.

The photoresist composition may also include one or more non-solvent alkali-insoluble base materials, which are typically present in a combined amount of greater than 50 weight percent, based on total solids of the photoresist composition. The one or more non-solvent alkali-insoluble base materials, which may alternatively be referred to herein as a matrix material, may be polymeric or non-polymeric. Suitable alkali-insoluble base materials will be apparent to the person of skill in the art and based on the description provided herein. In some embodiments, the alkali-insoluble base material does not include a phenolic hydroxyl group, such as a phenolic hydroxyl group-containing novolac resin. In some embodiments, the alkali-insoluble base material does not include a carboxylic acid group. In some embodiments, the alkali-insoluble base material may include a phenolic hydroxyl group and/or a carboxylic acid group provided alkali insolubility of the base material is maintained. As noted above, the matrix material may also be a polymer that includes a repeating unit derived from the polymerizable compounds described herein.

To determine if a particular base material is alkali insoluble, the base material may be subjected to solubility testing with an aqueous alkali developer solution such as 0.26 normal (N) aqueous tetramethylammonium hydroxide (TMAH). The alkali solubility may, for example, be determined using the following method. A film of the base material may be applied to the surface of a Si substrate by spin-coating and an initial film thickness measured. The film of the base material may be immersed in 0.26 N TMAH aqueous solution at room temperature for 60 seconds, followed by DI water rinse and air drying, which are typical development conditions, and then the thickness of the film is measured again. Alkali insolubility is indicated by a change in thickness of less than 2 nanometers (nm), preferably less than 1 nm, less than 0.5 nm, less than 0.1 nm, or 0 nm.

In some embodiments, the base material may comprise a polymer, a metal-containing material, or a combination thereof. It is to be understood that “base material” does not define the material as being basic (e.g., the base material is not necessarily basic according to the definition of acid/base chemistry).

The polymer of the photoresist composition may be a homopolymer or a copolymer that includes two or more structurally different repeating units. For example, the polymer may include one or more repeating units that include a functional group selected from a hydroxyaryl group, an acid-labile group, a base-solubilizing group, a lactone-containing group, a sultone-containing group, a polar group, a crosslinkable group, a crosslinking group, or the like, or a combination thereof. As noted above, the polymer may also include a repeating unit derived from the polymerizable compounds described herein.

In one or more embodiments, the polymer may include a repeating unit formed from a monomer that includes an acid-labile group. Suitable acid-labile groups include, for example, tertiary ester, acetal, ketal, and tertiary ether groups.

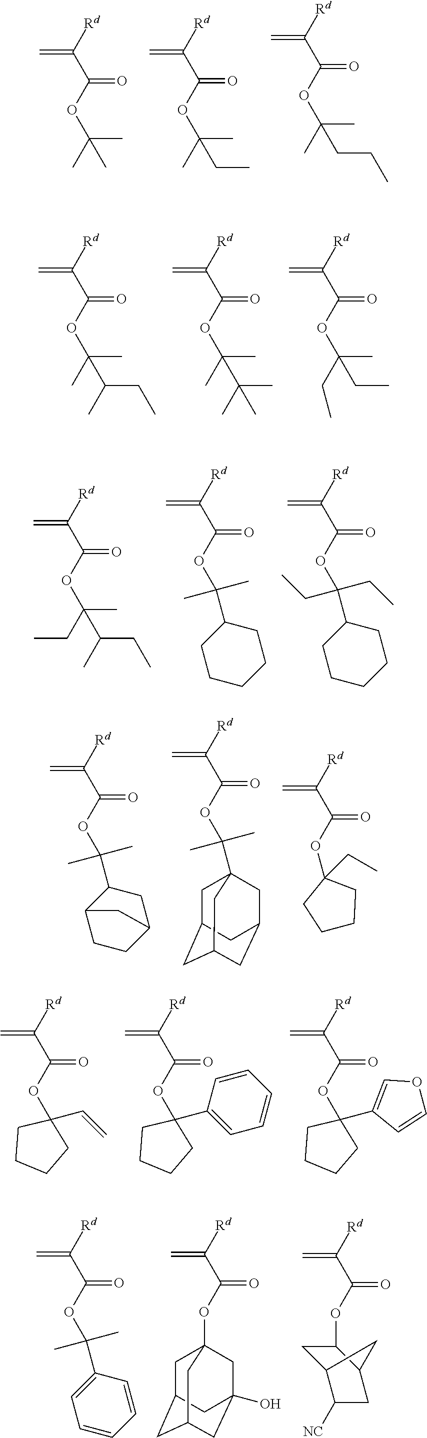

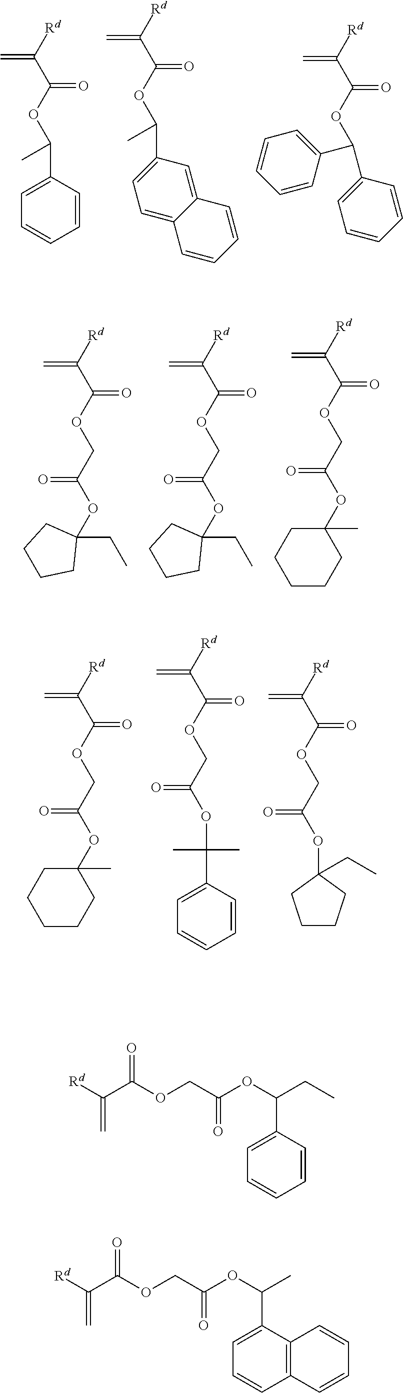

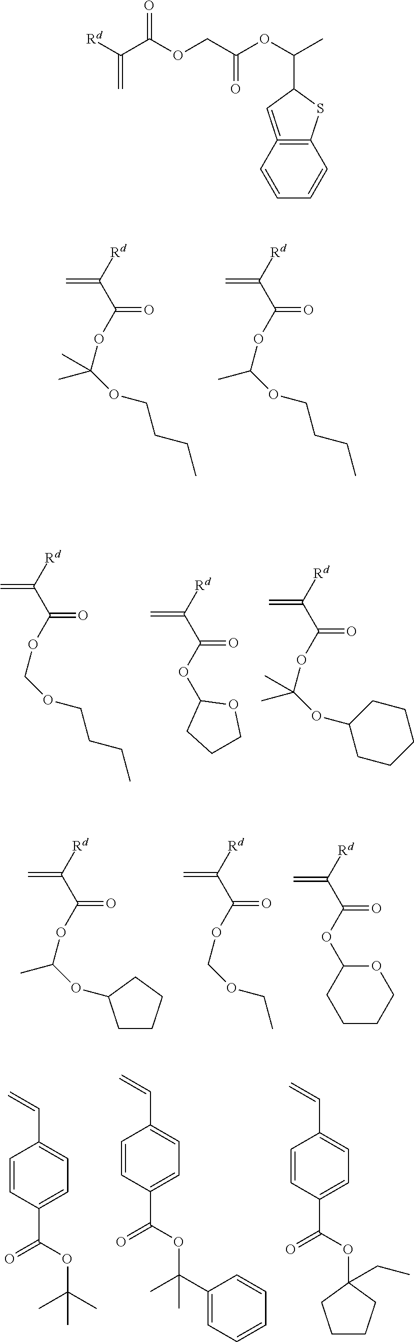

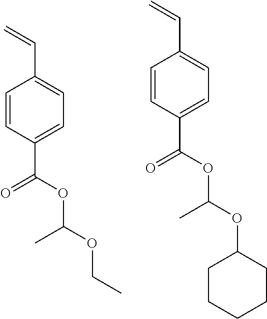

wherein R^{d} is hydrogen, halogen (e.g., F, Cl, Br, I), substituted or unsubstituted C₁₋₆ alkyl, or substituted or unsubstituted C₃₋₆ cycloalkyl.

When a repeating unit having an acid-labile group is present in the polymer, it is typically present in an amount from 25 to 75 mol %, more typically from 25 to 50 mol %, still more typically from 30 to 50 mol %, based on total repeating units in the polymer.

In some embodiments, the polymer may include a repeating unit derived from one or more lactone-containing monomers. Suitable lactone-containing monomers include, for example:

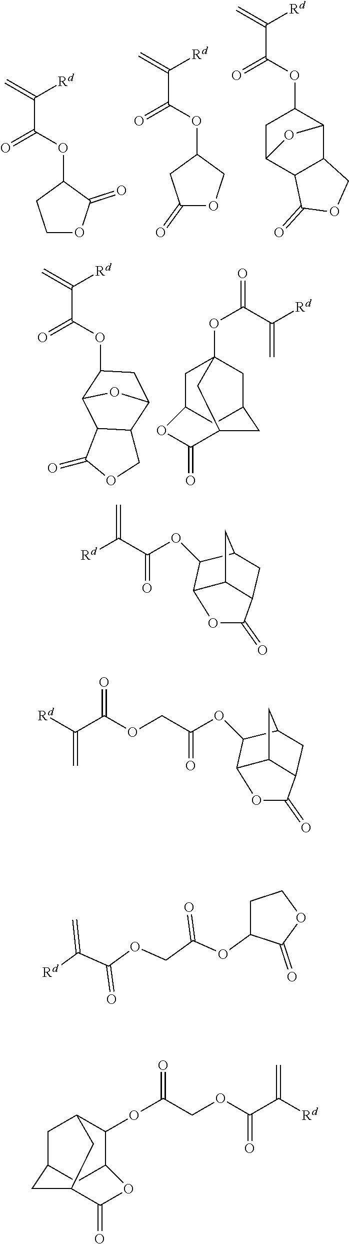

wherein R^{d} is hydrogen, halogen (e.g., F, Cl, Br, I), substituted or unsubstituted C₁₋₆ alkyl, or substituted or unsubstituted C₃₋₆ cycloalkyl.

When a repeating unit derived from one or more lactone-containing monomers is present in the polymer, it is typically present in an amount from 0.5 to 75 mol %, more typically from 1 to 50 mol %, still more typically from 5 to 50 mol %, based on total repeating units in the polymer.

In some embodiments, the polymer may include a repeating unit having a base-solubilizing group and/or having a pKa of less than or equal to 12. Exemplary base-solubilizing groups may comprise a fluoroalcohol group, a carboxylic acid group, a carboxamide group, a sulfonamide group, or a sulfonimide group.

Non-limiting examples of monomers including a base-solubilizing include the following:

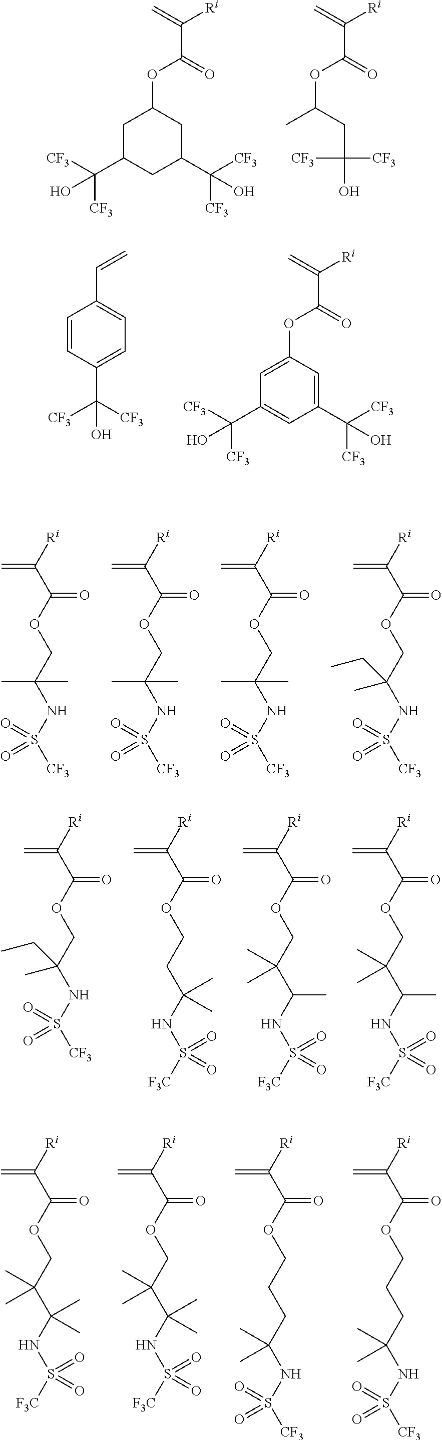

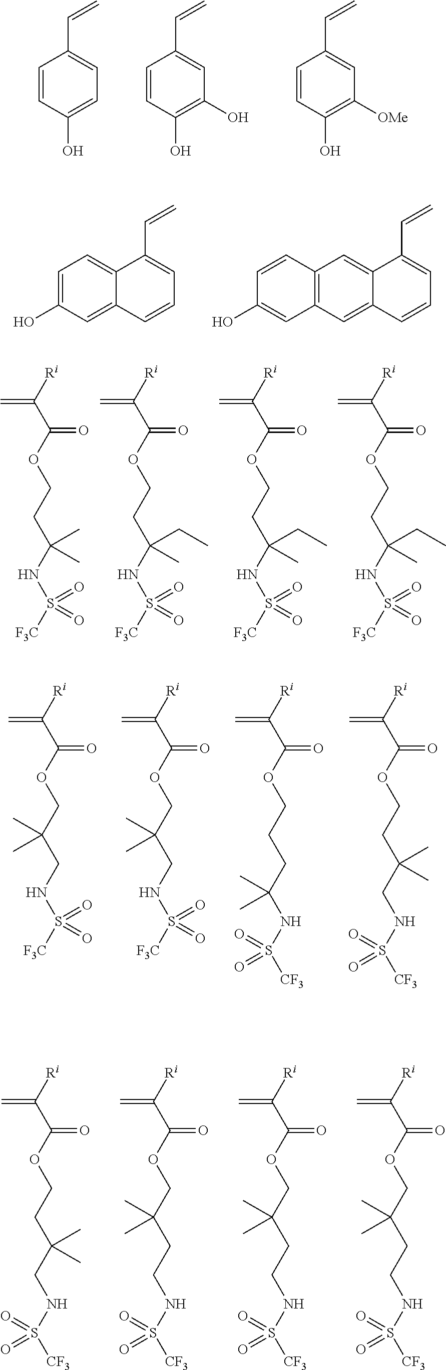

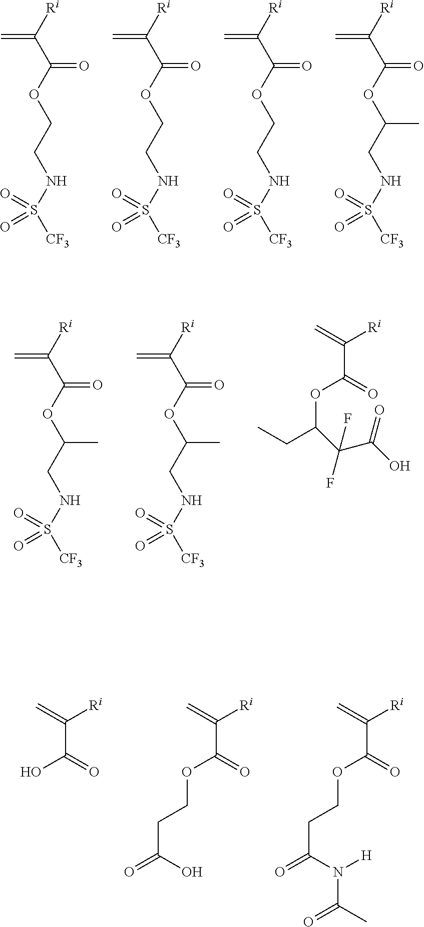

wherein Rⁱ is hydrogen, halogen (e.g., F, Cl, Br, I), substituted or unsubstituted C₁₋₆ alkyl, or substituted or unsubstituted C₃₋₆ cycloalkyl.

When a repeating unit having a base-solubilizing group and/or having a pKa of less than or equal to 12 is present in the polymer, it is typically present in an amount from 0.5 to 30 mol %, more typically from 15 to 25 mol %, still more typically from 5 to 10 mol %, based on total repeating units in the polymer.

The polymer may further optionally include one or more aromatic group-containing repeating units. For example, such repeating units may include one or more of the following:

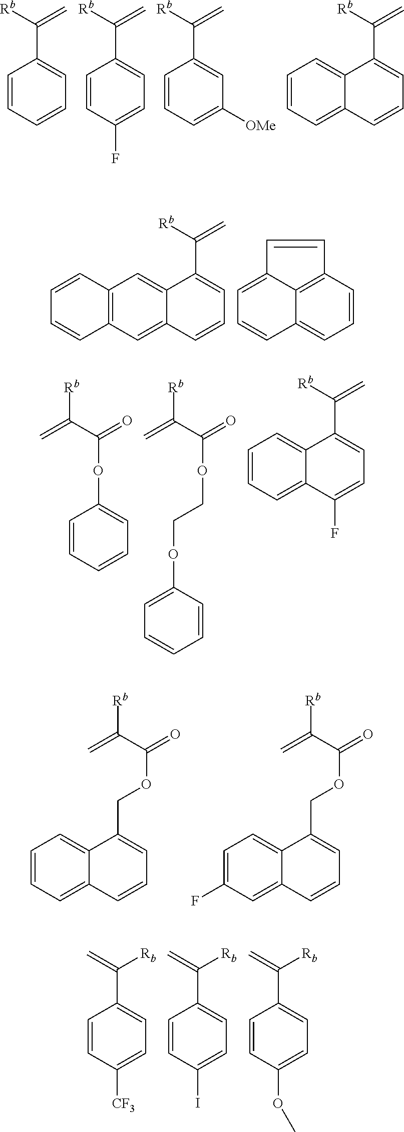

wherein R^{b} is hydrogen, halogen (e.g., F, Cl, Br, I), substituted or unsubstituted C₁₋₆ alkyl, or substituted or unsubstituted C₃₋₆ cycloalkyl.

When present, the polymer typically comprises an aromatic group-containing repeating unit in an amount from 1 to 80 mol %, more typically from 5 to 75 mol %, still more typically from 5 to 50 mol %, based on total repeating units in the polymer.

In some embodiments, the polymer may optionally include a repeating unit derived from an acetal monomer that does not include an ester acetal, such as a monomer of the following structures:

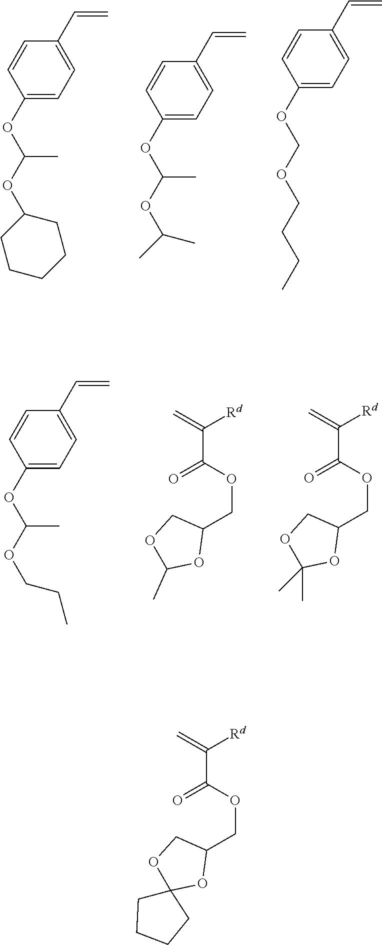

wherein R^{d} is hydrogen, fluorine, cyano, substituted or unsubstituted C₁₋₁₀ alkyl.

When present, polymer typically comprises a repeating unit having an acetal monomer that does not include an ester acetal in an amount from 1 to 80 mol %, more typically from 5 to 75 mol %, still more typically from 5 to 50 mol %, based on total repeating units in the polymer.

The polymer may further optionally include one or more additional repeating units. The additional repeating units may be, for example, one or more additional units for purposes of adjusting properties of the photoresist composition, such as etch rate and solubility. Exemplary additional units may include those derived from one or more of (meth)acrylate, vinyl aromatic, vinyl ether, vinyl ketone, and/or vinyl ester monomers. The one or more additional repeating units, if present in the polymer, may be used in an amount of up to 50 mol %, typically from 3 to 50 mol %, based on total repeating units of the polymer.

Non-limiting exemplary polymers of the non-solvent alkali-insoluble base material include one or more of the following:

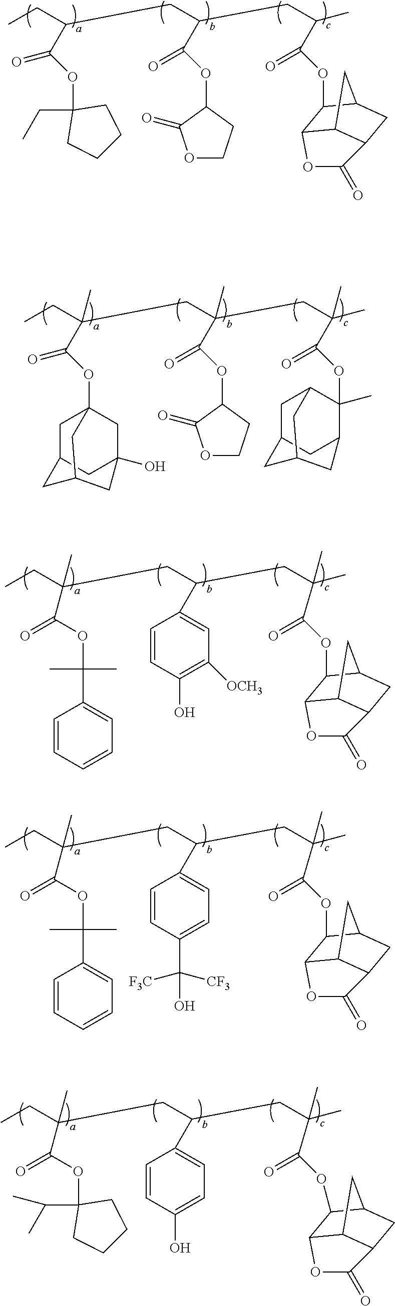

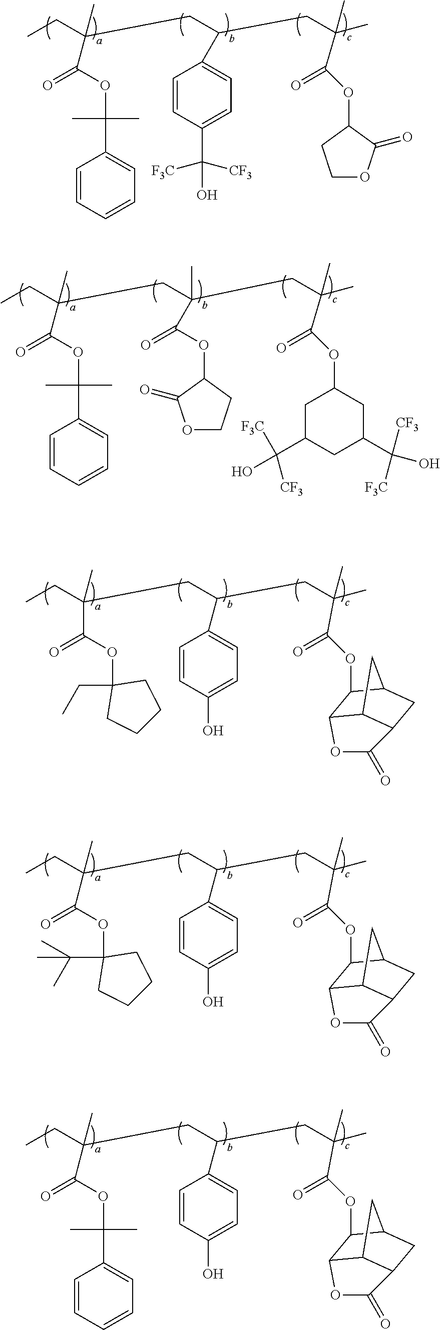

wherein a, b, and c represent the mole fractions for the respective repeating units of the polymer and a+b+c=1. It is to be understood that the mole fractions of a, b, and c are selected such that the polymer is alkali insoluble.

In some embodiment, and as described in further detail hereinabove, the non-solvent alkali insoluble base material may be a polymer that further includes a repeating unit that is derived from the inventive polymerizable compound. For example, a repeating unit of the exemplary polymers described above may be substituted for a repeating unit that is derived from the inventive polymerizable compound described herein, or, for example, an additional repeating unit that is derived from the inventive polymerizable compound as described herein may be added to the exemplary polymers described above.

In some embodiments, the non-solvent alkali-insoluble base material may include a chain-scissionable polymer, an unzipping polymer, or a combination thereof.

Chain-scissionable polymers can undergo chain scission reactions under suitable conditions. Any suitable chain-scissionable polymer may be used. Exemplary direct photolysis, chain-scissionable polymers include, for example, copolymers of α-substituted styrene(s) and substituted α-halogen acrylates, for example, α-methylstyrene/methyl-α-chloroacrylate copolymer, 2-trifluoroethyl-α-chloroacrylate/α-methyl-4-fluorostyrene copolymer, or the like, or a combination thereof.

Unzipping polymers include polymers having unzipping polymer end group upon suitable stimulation (photoinduced or chemically induced stimulus) which triggers breaking the polymer backbone into smaller parts. Typically, the unzipping polymer is chosen such that stimulating a first chemical modification or degradation event triggers an unzipping effect that is partial or total. Any suitable unzipping polymer may be used.

The polymer typically has a M_{w} from 1,000 to 200,000 Da, preferably from 10,000 to 150,000 Da, more preferably 15,000 to 150,000 Da, and still more preferably from 25,000 to 150,000 Da or from 50,000 to 150,000 Da. The PDI of the polymer is typically from 1.1 to 3, and more typically from 1.1 to 2. Molecular weight values are determined by GPC using polystyrene standards.

The polymer may be prepared using any suitable method(s) in the art. For example, one or more monomers corresponding to the repeating units described herein may be combined, or fed separately, using suitable solvent(s) and initiator, and polymerized in a reactor. For example, the polymers may be obtained by polymerization of the respective monomers under any suitable conditions, such as by heating at an effective temperature, irradiation with actinic radiation at an effective wavelength, or a combination thereof.

In some embodiments, the one or more non-solvent alkali-insoluble base materials may be a metal-containing material. Exemplary metal-containing materials include metalorganic resists (e.g., photoinduced crosslinkable metalorganic resists, or the like), metal oxide resists, or the like, or a combination thereof. In some embodiment, the metal-containing material may include Sn, Zr, Hf, Si, Ge, Se, Cr, Mo, W, V, Nb, Ta, P, Sb, Ti, Ce, Ru, Sb, Y, Ga, Cr, Fe, Co, Ru, Al, In, Sc, Bi, La, Ce, Pr, Nd, Pm, Sm, Eu, Gd, Tb, Dy, Ho, Er, Tm, Yb, Lu, Zn, Co, Ni, Mn, Mg, Ca, Sr, Ba, or a combination thereof. Typically, the metal-containing material may include Sn, Zr, Hf, Si, Ge, Se, or a combination thereof.

The one or more non-solvent alkali-insoluble base materials is/are present in a combined amount of greater than 50 wt % based on total solids of the photoresist composition. For example, the one or more non-solvent alkali-insoluble base materials may be present in a combined amount from 50 wt % to 99 wt %, typically from 60 wt % to 95 wt %, or from 70 wt % to 90 wt %, based on total solids of the photoresist composition.

The photoresist composition further includes a solvent for dissolving the components of the composition and to facilitate its coating on a substrate. Preferably, the solvent is an organic solvent conventionally used in the manufacture of electronic devices. Suitable solvents include, for example: aliphatic hydrocarbons such as hexane and heptane; aromatic hydrocarbons such as toluene and xylene; halogenated hydrocarbons such as dichloromethane, 1,2-dichloroethane and 1-chlorohexane; alcohols such as methanol, ethanol, 1-propanol, iso-propanol, tert-butanol, 2-methyl-2-butanol, 4-methyl-2-pentanol, and diacetone alcohol (4-hydroxy-4-methyl-2-pentanone) (DAA); propylene glycol monomethyl ether (PGME); ethers such as diethyl ether, tetrahydrofuran, 1,4-dioxane, and anisole; ketones such as acetone, methyl ethyl ketone, methyl iso-butyl ketone, 2-heptanone, and cyclohexanone (CHO); esters such as ethyl acetate, n-butyl acetate, propylene glycol monomethyl ether acetate (PGMEA), ethyl lactate (EL), hydroxyisobutyrate methyl ester (HBM), and ethyl acetoacetate; lactones such as gamma-butyrolactone (GBL) and epsilon-caprolactone; lactams such as N-methyl pyrrolidone; nitriles such as acetonitrile and propionitrile; cyclic or non-cyclic carbonate esters such as propylene carbonate, dimethyl carbonate, ethylene carbonate, propylene carbonate, diphenyl carbonate, and propylene carbonate; polar aprotic solvents such as dimethyl sulfoxide and dimethyl formamide; water; or a combination thereof. Of these, preferred solvents are PGME, PGMEA, EL, GBL, HBM, CHO, DAA, or a combination thereof.

The total solvent content (i.e., cumulative solvent content for all solvents) in the photoresist compositions is typically from 40 to 99 wt %, for example, from 60 to 99 wt %, or from 85 to 99 wt %, based on total solids of the photoresist composition. The desired solvent content will depend, for example, on the desired thickness of the coated photoresist layer and coating conditions.

In some aspects, the photoresist composition may further include a material that comprises one or more base-labile groups (a “base-labile material”). As referred to herein, base-labile groups are functional groups that can undergo cleavage reaction to provide polar groups such as hydroxyl, carboxylic acid, sulfonic acid, and the like, in the presence of an aqueous alkaline developer after exposure and post-exposure baking steps. The base-labile group will not react significantly (e.g., will not undergo a bond-breaking reaction) prior to a development step of the photoresist composition that comprises the base-labile group. Thus, for instance, a base-labile group will be substantially inert during pre-exposure soft-bake, exposure, and post-exposure bake steps. By “substantially inert” it is meant that ≤5%, typically ≤1%, of the base-labile groups (or moieties) will decompose, cleave, or react during the pre-exposure soft-bake, exposure, and post-exposure bake steps. The base-labile group is reactive under typical photoresist development conditions using, for example, an aqueous alkaline photoresist developer such as a 0.26 normal (N) aqueous solution of tetramethylammonium hydroxide (TMAH). For example, a 0.26 N aqueous solution of TMAH may be used for single puddle development or dynamic development, e.g., where the 0.26 N TMAH developer is dispensed onto an imaged photoresist layer for a suitable time such as 10 to 120 seconds (s). An exemplary base-labile group is an ester group, typically a fluorinated ester group. Preferably, the base-labile material is substantially not miscible with and has a lower surface energy than the first and/or second polymers and other solid components of the photoresist composition. When coated on a substrate, the base-labile material can thereby segregate from other solid components of the photoresist composition to a top surface of the formed photoresist layer.

In some aspects, the base-labile material may be a polymeric material, also referred to herein as a base-labile polymer, which may include one or more repeating units comprising one or more base-labile groups. For example, the base-labile polymer may comprise a repeating unit comprising 2 or more base-labile groups that are the same or different. A preferred base-labile polymer includes at least one repeating unit comprising 2 or more base-labile groups, for example a repeating unit comprising 2 or 3 base-labile groups.

The base-labile polymer may be prepared using any suitable methods in the art, including those described herein for the first and second polymers. For example, the base-labile polymer may be obtained by polymerization of the respective monomers under any suitable conditions, such as by heating at an effective temperature, irradiation with actinic radiation at an effective wavelength, or a combination thereof. Additionally, or alternatively, one or more base-labile groups may be grafted onto the backbone of a polymer using suitable methods.

In some aspects, the base-labile material is a single molecule comprising one more base-labile ester groups, preferably one or more fluorinated ester groups. The base-labile materials that are single molecules typically have a M_{w} in the range from 50 to 1,500 Da.

When present, the base-labile material is typically present in the photoresist compositions in an amount of from 0.01 to 10 wt % or 2 to 7 w %, typically from 1 to 5 wt %, based on total solids of the photoresist composition.

Additionally, or alternatively, to the base-labile polymer, the photoresist compositions may further include one or more polymers in addition to and different from the non-solvent alkali-insoluble base material as described above. For example, the photoresist compositions may include an additional polymer as described above but different in composition. Additionally, or alternatively, the one or more additional polymers may include those well known in the photoresist art, for example, those chosen from polyacrylates, polyvinylethers, polyesters, polynorbornenes, polyacetals, polyethylene glycols, polyamides, polyacrylamides, polyphenols, novolacs, styrenic polymers, polyvinyl alcohols, or combinations thereof.

The photoresist composition may further include one or more additional, optional additives. For example, optional additives may include actinic and contrast dyes, anti-striation agents, plasticizers, speed enhancers, sensitizers, photo-decomposable quenchers (PDQ) (and, also known as photo-decomposable bases), basic quenchers, thermal acid generators, surfactants, and the like, or combinations thereof. If present, the optional additives are typically present in the photoresist compositions in an amount of from 0.01 to 10 wt %, based on total solids of the photoresist composition.

PDQs generate a weak acid upon irradiation. The acid generated from a photo-decomposable quencher is not strong enough to react rapidly with acid-labile groups that are present in the resist matrix. Exemplary photo-decomposable quenchers include, for example, photo-decomposable cations, and preferably those also useful for preparing strong acid generator compounds, paired with an anion of a weak acid (pKa>1) such as, for example, an anion of a C₁₋₂₀ carboxylic acid or C₁₋₂₀ sulfonic acid. Exemplary carboxylic acids include formic acid, acetic acid, propionic acid, tartaric acid, succinic acid, cyclohexanecarboxylic acid, benzoic acid, salicylic acid, and the like. Exemplary sulfonic acids include p-toluene sulfonic acid, camphor sulfonic acid and the like. In a preferred embodiment, the photo-decomposable quencher is a photo-decomposable organic zwitterion compound such as diphenyliodonium-2-carboxylate.

The photo-decomposable quencher may be in non-polymeric or polymer-bound form. When in polymeric form, the photo-decomposable quencher is present in polymerized units on the first polymer or second polymer. The polymerized units containing the photo-decomposable quencher are typically present in an amount from 0.1 to 30 mole %, preferably from 1 to 10 mole % and more preferably from 1 to 2 mole %, based on total repeating units of the polymer.

Exemplary basic quenchers include, for example, linear aliphatic amines such as tributylamine, trioctylamine, triisopropanolamine, tetrakis(2-hydroxypropyl)ethylenediamine:n-tert-butyldiethanolamine, tris(2-acetoxy-ethyl) amine, 2,2′,2″,2′″-(ethane-1,2-diylbis(azanetriyl))tetraethanol, 2-(dibutylamino)ethanol, and 2,2′,2″-nitrilotriethanol; cyclic aliphatic amines such as 1-(tert-butoxycarbonyl)-4-hydroxypiperidine, tert-butyl 1-pyrrolidinecarboxylate, tert-butyl 2-ethyl-1H-imidazole-1-carboxylate, di-tert-butyl piperazine-1,4-dicarboxylate, and N-(2-acetoxy-ethyl)morpholine; aromatic amines such as pyridine, di-tert-butyl pyridine, and pyridinium; linear and cyclic amides and derivatives thereof such as N,N-bis(2-hydroxyethyl)pivalamide, N,N-diethylacetamide, N¹,N¹,N³,N³-tetrabutylmalonamide, 1-methylazepan-2-one, 1-allylazepan-2-one, and tert-butyl 1,3-dihydroxy-2-(hydroxymethyl)propan-2-ylcarbamate; ammonium salts such as quaternary ammonium salts of sulfonates, sulfamates, carboxylates, and phosphonates; imines such as primary and secondary aldimines and ketimines; diazines such as optionally substituted pyrazine, piperazine, and phenazine; diazoles such as optionally substituted pyrazole, thiadiazole, and imidazole; and optionally substituted pyrrolidones such as 2-pyrrolidone and cyclohexyl pyrrolidine.

The basic quenchers may be in non-polymeric or polymer-bound form. When in polymeric form, the quencher may be present in repeating units of the polymer. The repeating units containing the quencher are typically present in an amount of from 0.1 to 30 mole %, preferably from 1 to 10 mole % and more preferably from 1 to 2 mole %, based on total repeating units of the polymer.

Exemplary surfactants include fluorinated and non-fluorinated surfactants and can be ionic or non-ionic, with non-ionic surfactants being preferable. Exemplary fluorinated non-ionic surfactants include perfluoro C₄ surfactants such as FC-4430 and FC-4432 surfactants, available from 3M Corporation; and fluorodiols such as POLYFOX PF-636, PF-6320, PF-656, and PF-6520 fluorosurfactants from Omnova. In an aspect, the photoresist composition further includes a surfactant polymer including a fluorine-containing repeating unit.

Patterning methods using the photoresist compositions of the invention will now be described. Suitable substrates on which the photoresist compositions can be coated include electronic device substrates. A wide variety of electronic device substrates may be used in the present invention, such as: semiconductor wafers; polycrystalline silicon substrates; packaging substrates such as multichip modules; flat panel display substrates; substrates for light emitting diodes (LEDs) including organic light emitting diodes (OLEDs); and the like, with semiconductor wafers being typical. Such substrates are typically composed of one or more of silicon, polysilicon, silicon oxide, silicon nitride, silicon oxynitride, silicon germanium, gallium arsenide, aluminum, sapphire, tungsten, titanium, titanium-tungsten, nickel, copper, and gold. Suitable substrates may be in the form of wafers such as those used in the manufacture of integrated circuits, optical sensors, flat panel displays, integrated optical circuits, and LEDs. Such substrates may be any suitable size. Typical wafer substrate diameters are 200 to 300 millimeters (mm), although wafers having smaller and larger diameters may be suitably employed according to the present invention. The substrates may include one or more layers or structures which may optionally include active or operable portions of devices being formed.

Typically, one or more lithographic layers such as a hardmask layer, for example, a spin-on-carbon (SOC), amorphous carbon, or metal hardmask layer, a CVD layer such as a silicon nitride (SiN), a silicon oxide (SiO), or silicon oxynitride (SiON) layer, an organic or inorganic underlayer, or combinations thereof, are provided on an upper surface of the substrate prior to coating a photoresist composition of the present invention. Such layers, together with an overcoated photoresist layer, form a lithographic material stack.

Optionally, a layer of an adhesion promoter may be applied to the substrate surface prior to coating the photoresist compositions. If an adhesion promoter is desired, any suitable adhesion promoter for polymer films may be used, such as silanes, typically organosilanes such as trimethoxyvinylsilane, triethoxyvinylsilane, hexamethyldisilazane, or an aminosilane coupler such as gamma-aminopropyltriethoxysilane. Particularly suitable adhesion promoters include those sold under the AP™ 3000, AP™ 8000, and AP™ 9000S designations, available from DuPont Electronics & Industrial (Marlborough, Massachusetts).

The photoresist composition may be coated on the substrate by any suitable method, including spin coating, spray coating, dip coating, doctor blading, or the like. For example, applying the layer of photoresist may be accomplished by spin coating the photoresist in solvent using a coating track, in which the photoresist is dispensed on a spinning wafer. During dispensing, the wafer is typically spun at a speed of up to 4,000 rotations per minute (rpm), for example, from 200 to 3,000 rpm, for example, from 1,000 to 2,500 rpm, for a period from 15 to 120 seconds to obtain a layer of the photoresist composition on the substrate. It will be appreciated by those skilled in the art that the thickness of the coated layer may be adjusted by changing the spin speed and/or the total solids of the composition. A photoresist composition layer formed from the compositions of the invention typically has a dried layer thickness from 1 nanometer (nm) to 120 micrometers (μm), preferably from greater than 5 nm to 110 μm, and more preferably from 6 to 100 μm. In some embodiments, the photoresist composition layer formed from the compositions may have a dried layer thickness from 10 nm to 5 μm, or from 3 to 20 μm.

The photoresist composition is typically next soft-baked to minimize the solvent content in the layer, thereby forming a tack-free coating and improving adhesion of the layer to the substrate. The soft bake is performed, for example, on a hotplate or in an oven, with a hotplate being typical. The soft bake temperature and time will depend, for example, on the photoresist composition and thickness. The soft bake temperature is typically from 80 to 170° C., and more typically from 90 to 150° C. The soft bake time is typically from 10 seconds to 20 minutes, more typically from 1 to 10 minutes, and still more typically from 1 to 2 minutes. The heating time can be readily determined by one of ordinary skill in the art based on the ingredients of the composition.

The photoresist layer is next pattern-wise exposed to activating radiation to create a difference in solubility between exposed and unexposed regions. Reference herein to exposing a photoresist composition to radiation that is activating for the composition indicates that the radiation can form a latent image in the photoresist composition. The exposure is typically conducted through a patterned photomask that has optically transparent and optically opaque regions corresponding to regions of the resist layer to be exposed and unexposed, respectively. Such exposure may, alternatively, be conducted without a photomask in a direct writing method, typically used for e-beam lithography. The activating radiation typically has a wavelength of sub-400 nm, sub-300 nm or sub-200 nm, with 248 nm (KrF), 193 nm (ArF), 13.5 nm (EUV) wavelengths or e-beam lithography being preferred. Preferably, the activating radiation is 248 nm radiation. The methods find use in immersion or dry (non-immersion) lithography techniques. The exposure energy is typically from 1 to 200 millijoules per square centimeter (mJ/cm²), preferably from 10 to 100 mJ/cm² and more preferably from 20 to 50 mJ/cm², dependent upon the exposure tool and components of the photoresist composition.

Following exposure of the photoresist layer, a postexposure bake (PEB) of the exposed photoresist layer is performed. The PEB can be conducted, for example, on a hotplate or in an oven, with a hotplate being typical. Conditions for the PEB will depend, for example, on the photoresist composition and layer thickness. The PEB is typically conducted at a temperature from 70 to 150° C., preferably from 75 to 120° C., and a time from 30 to 120 seconds. A latent image defined by the polarity-switched (exposed regions) and unswitched regions (unexposed regions) is formed in the photoresist.

The exposed photoresist layer is then developed with a suitable developer to selectively remove those regions of the layer that are soluble in the developer while the remaining insoluble regions form the resulting photoresist pattern relief image. In the case of a positive-tone development (PTD) process, the exposed regions of the photoresist layer are removed during development and unexposed regions remain. Conversely, in a negative-tone development (NTD) process, the exposed regions of the photoresist layer remain, and unexposed regions are removed during development. Application of the developer may be accomplished by any suitable method such as described above with respect to application of the photoresist composition, with spin coating being typical. The development time is for a period effective to remove the soluble regions of the photoresist, with a time of from 5 to 60 seconds being typical. Development is typically conducted at room temperature.

Suitable developers for a PTD process include aqueous base developers, for example, quaternary ammonium hydroxide solutions such as tetramethylammonium hydroxide (TMAH), preferably 0.26 normal (N) TMAH, tetraethylammonium hydroxide, tetrabutylammonium hydroxide, sodium hydroxide, potassium hydroxide, sodium carbonate, potassium carbonate, and the like. Suitable developers for an NTD process are organic solvent-based, meaning the cumulative content of organic solvents in the developer is 50 wt % or more, typically 95 wt % or more, 98 wt % or more, or 100 wt %, based on total weight of the developer. Suitable organic solvents for the NTD developer include, for example, those chosen from ketones, esters, ethers, hydrocarbons, and mixtures thereof. The developer is typically 2-heptanone or n-butyl acetate.

A coated substrate may be formed from the photoresist compositions of the invention. Such a coated substrate includes: (a) a substrate having one or more layers to be patterned on a surface thereof; and (b) a layer of the photoresist composition over the one or more layers to be patterned.

The photoresist pattern may be used, for example, as an etch mask, thereby allowing the pattern to be transferred to one or more sequentially underlying layers by known etching techniques, typically by dry etching such as reactive ion etching. The photoresist pattern may, for example, be used for pattern transfer to an underlying hardmask layer which, in turn, is used as an etch mask for pattern transfer to one or more layers below the hardmask layer. If the photoresist pattern is not consumed during pattern transfer, it may be removed from the substrate by known techniques, for example, oxygen plasma ashing. The photoresist compositions may, when used in one or more such patterning processes, be used to fabricate semiconductor devices such as memory devices, processor chips (CPUs), graphics chips, optoelectronic chips, LEDs, OLEDs, as well as other electronic devices.

The invention is further illustrated by the following non-limiting examples.

### EXAMPLES

All reactions were carried out under ambient atmospheric conditions. All chemicals were used directly from the supplier. Nuclear magnetic resonance (NMR) spectra for all compounds were obtained on a 500 MHz spectrometer unless otherwise noted. The chemical shifts are reported in δ (parts per million, ppm) values relative to internal deuterated chloroform residual signal. Multiplicities are indicated by s (singlet), d (doublet), t (triplet), m (multiplet), dd (doublet of doublets), dt (doublet of triplets), tt (triplet of triplets), br (broad singlet).

### Synthesis of sodium 4-carboxy-2-hydroxybenzenesulfonate

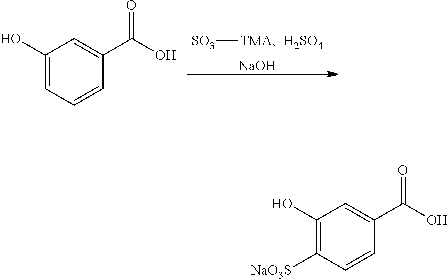

Sulfur trioxide-trimethylamine (SO₃-TMA) (0.7 grams (g)) was added to a solution of 3-hydroxybenzoic acid (20.0 g) in H₂SO₄ (40 milliliters (mL)) at room temperature. The reaction mixture was heated to 100° C., stirred for 16 hours, cooled to room temperature, and diluted with ice water followed by 25% aqueous NaOH. The resulting solid was stirred for 30 minutes, filtered, suspended in toluene (40 mL) and distilled. This process was repeated two more times and then the solid was washed with acetone (3×50 mL) and dried to afford sodium 4-carboxy-2-hydroxybenzenesulfonate (25 g, 72%) as a light green solid. ESI-MS: [M-Na]⁻: 217.13

### Synthesis of sodium 2-hydroxy-4-((2-(methacryloyloxy)ethoxy)carbonyl) benzenesulfonate

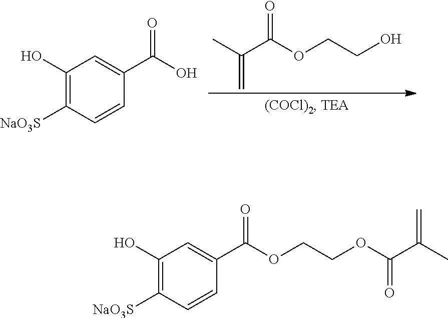

Phosgene (3.96 g) in dimethylformamide (DMF) (3.0 mL) was added to a solution of sodium 4-carboxy-2-hydroxybenzenesulfonate (3.0 g) in 1,2-dimethoxyethane (60 mL) at 0° C., warmed to room temperature, and stirred for 16 hours. The crude mixture as concentrated under argon atmosphere followed by the addition of hydroxyethylmethacrylate (15 mL). The reaction mixture was cooled to 0° C., triethylamine (3.78 g) was added, and the reaction mixture was warmed to room temperature and stirred for 24 hours. Ethyl acetate was added, and the organic layer was washed with water then brine. The organic layer was concentrated under reduced pressure and the resulting solid washed with methyl tert-butyl ether (MTBE). Further purification was achieved via silica gel column chromatography (gradient 0% to 10% methanol in dichloromethane) to afford sodium 2-hydroxy-4-((2-(methacryloyloxy)ethoxy)carbonyl) benzenesulfonate (0.45 g, 10%) as an off-white solid. ESI-MS: [M-Na]⁻: 329.43 ¹H NMR (400 MHz, DMSO-d₆): δ 10.64 (s, 1H), 7.57 (d, J=8.0 Hz, 1H), 7.38 (dd, J=8.0, 1.6 Hz, 1H), 7.29 (d, J=1.6 Hz, 1H), 6.02 (s, 1H), 5.68 (t, J=1.6 Hz, 1H), 4.52-4.49 (m, 2H), 4.45-4.43 (m, 2H), 1.86 (s, 3H).

### Synthesis of triphenylsulfonium 2-hydroxy-4-((2-(methacryloyloxy)ethoxy)carbonyl) benzenesulfonate (M1)

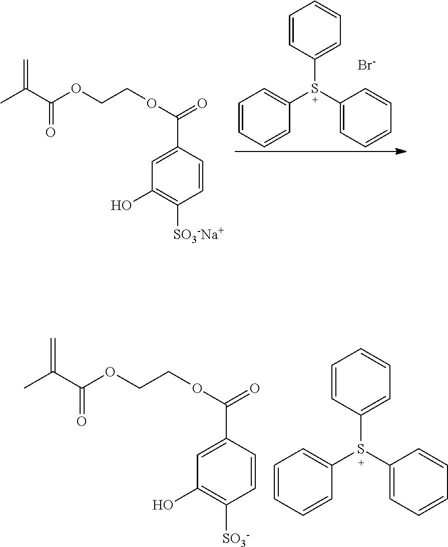

Triphenylsulfonium bromide (5.8 g) and sodium 2-hydroxy-4-((2-(methacryloyloxy)ethoxy)carbonyl) benzenesulfonate (5.1 g) were dissolved in dichloromethane (116 mL) and water (58 mL). The mixture was vigorously stirred for 4 hours, the organic layer was separated and washed with water (5×100 mL), and then the organic layer was concentrated under reduced pressure to afford triphenylsulfonium 2-hydroxy-4-((2-(methacryloyloxy)ethoxy)carbonyl) benzenesulfonate M1 (6.5 g, 66%) as a thick solid. ESI-MS: [M]⁻: 329.22, [M]⁺: 263.27 ¹H NMR (400 MHz, DMSO-d₆): δ 10.65 (s, 1H), 7.88-7.76 (m, 15H), 7.57 (d, J=8.0 Hz, 1H), 7.38 (dd, J=8.0, 1.6 Hz, 1H), 7.29 (d, J=1.6 Hz, 1H), 6.03-6.02 (m, 1H), 5.68-5.67 (m, 1H), 4.52-4.49 (m, 2H), 4.45-4.43 (m, 2H), 1.86 (s, 3H).

### Synthesis of tetraethylammonium 4-((2,6-diiodo-4-vinylphenoxy)carbonyl)-2 hydroxybenzenesulfonate

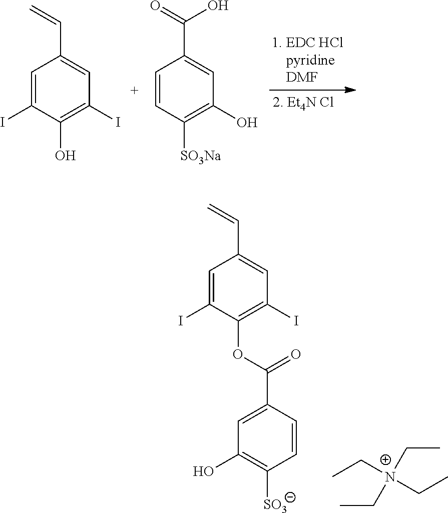

A mixture of 2,6-diiodo-4-vinylphenol (9.03 g, 24.3 mmol), sodium 4-carboxy-2-hydroxybenzenesulfonate (7.00 g, 29.2 mmol), and pyridine (2.35 mL, 29.2 mmol) were dissolved in N,N-dimethylformamide (70 mL). 1-(3-Dimethylaminopropyl)-3-ethylcarbodiimide hydrochloride [EDC HCl] (5.59 g, 29.2 mmol) was added portion-wise at room temperature and the reaction was stirred for 2 hours. The reaction mixture was poured into a solution of tetraethylammonium chloride (12.0 g, 72.7 mmol) in water (100 mL). This solution was extracted with dichloromethane (150 mL×2). Combined organic layers were washed with water (300 mL×3). The organic layer was dried over filter paper and was concentrated under reduced pressure. The resulting residue was recrystallized from acetone/methyl tert-butyl ether at 0° C. The resulting solid was filtered and generously rinsed with methyl tert-butyl ether. The solid was dried on filter paper under vacuum at room temperature to afford tetraethylammonium 4-((2,6-diiodo-4-vinylphenoxy)carbonyl)-2 hydroxybenzenesulfonate (9.54 g, 56%). ¹H NMR (500 MHz, DMSO-d₆) δ 10.77 (s, 1H), 8.05 (s, 2H), 7.72 (d, 1H), 7.64 (d, 1H), 7.53 (s, 1H), 6.69 (dd, 1H), 5.99 (d, 1H), 3.20 (q, 8H), 1.16 (m, 12H).

### Synthesis of Bis(3,5-difluorophenyl)(4-fluorophenyl)sulfonium 4-((2,6-diiodo-4-vinylphenoxy)carbonyl)-2-hydroxybenzenesulfonate (M2)

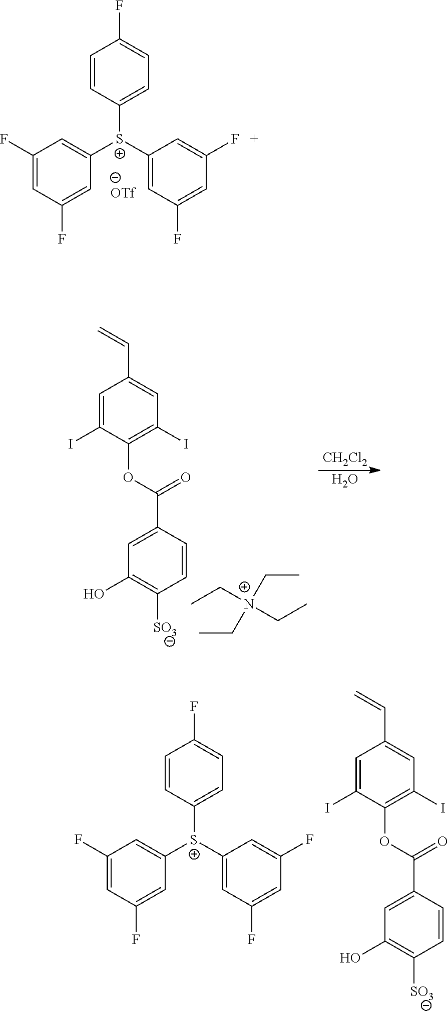

A mixture of tetraethylammonium 4-((2,6-diiodo-4-vinylphenoxy)carbonyl)-2 hydroxybenzenesulfonate (9.54 g, 13.6 mmol) and bis(3,5-difluorophenyl)(4-fluorophenyl)sulfonium trifluoromethanesulfonate (6.83 g, 13.6 mmol) was partitioned between dichloromethane (100 mL) and water (100 mL). The mixture was stirred at room temperature for 1 hour. The layers were separated and the organic layer was washed with saturated sodium chloride solution (100 mL×3) and water (100 mL×3). The organic layer was diluted to 300 mL with dichloromethane and washed with additional saturated sodium chloride solution (300 mL×2) and water (300 mL×3). The resulting organic layer was dried over filter paper and concentrated under reduced pressure. The residue was dissolved in acetone (70 mL) and methyl tert-butyl ether (600 mL) was added. The solution was cooled to 0° C. for 1 hour. The solid that precipitated was isolated by decanting the liquid and then dried under vacuum to produce 11.5 g of bis(3,5-difluorophenyl)(4-fluorophenyl)sulfonium 4-((2,6-diiodo-4-vinylphenoxy)carbonyl)-2-hydroxybenzenesulfonate (11.5 g, 91%) M2. ¹H NMR (500 MHz, Acetone-d₆) δ 10.91 (s, 1H), 8.46-8.15 (m, 2H), 8.04 (s, 2H), 7.92-7.74 (m, 5H), 7.74-7.51 (m, 6H), 6.73 (dd, J=17.6, 11.0 Hz, 1H), 5.96 (d, J=17.6 Hz, 1H), 5.39 (d, J=11.0 Hz, 1H). ¹⁹F NMR (470 MHz, Acetone) δ −101.9 (m), −103.9 (m).

### Polymer Synthesis

The following monomers were used to prepare the polymers P1 to P4 and the comparative polymer P5.

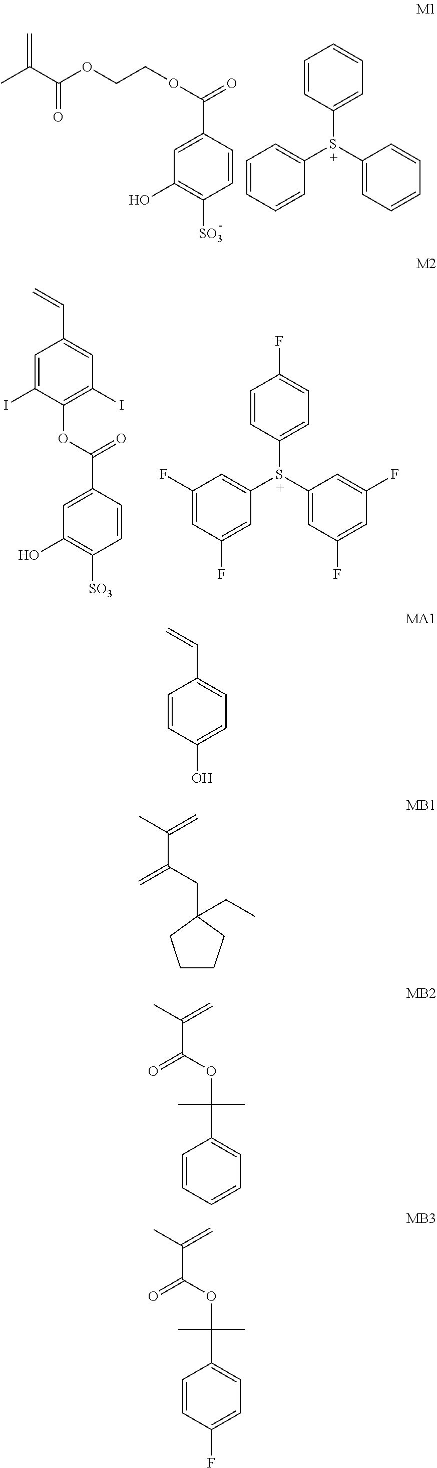

### Synthesis of Polymer P1

Polymer P1 was prepared from the monomers MA1, MB2, and M1 at a molar feed ratio of 40:50:10. A feed solution was prepared by dissolving MA1 (4.86 g, 40.5 mmol), MB2 (10.34 g, 50.61 mmol), M1 (6.00 g, 10.12 mmol), and V-601 initiator (2.94 g, 12.65 mmol; dimethyl 2,2′-azobis(2-methylpropionate), obtained from Wako Pure Chemical Industries, Ltd.) in propylene glycol monomethyl ether (PGME, 30.58 g).

The polymerization was performed in a flask fitted with a condenser and a thermometer to monitor the reaction in the flask. The reactor was charged with 30.0 g of PGMEA and heated to 80° C. The feed solution prepared above was fed into the flask using syringe pumps over 4 hours. Following the addition, the contents were then stirred for an additional 2 hours. The contents were subsequently cooled to room temperature. The polymer solution was added slowly to a large excess of isopropanol (500 ml) to precipitate the polymer. The resulting polymer precipitate was isolated by filtration and dried. The crude polymer was dissolved in THF to make 40% solid solution which was slowly poured into water and the precipitated polymer P1 was filtered and dried.

Each of the polymers P2 to P5 in Table 1 was prepared using a similar procedure to that described for the preparation of polymer P1, except that the monomers and molar feed ratios as specified in Table 1 were used. Also reported in Table 1 are the molecular weight M_{w} (in Daltons, Da, as determined by GPC) and the polydispersity index (PDI, M_{w}/Mₙ) for each of polymers P1 to P5.

TABLE 1

| | Monomer 1 | Monomer 2 | Monomer 3 | M_{w} (Da), |
|---|---|---|---|---|
| Polymer | (mol %) | (mol %) | (mol %) | PDI |
| P1 | MA1 (40) | MB2 (50) | M1 (10) | 5413, 1.48 |
| P2 | MA1 (35) | MB3 (50) | M1 (15) | 4408, 1.32 |
| P3 | MA1 (40) | MB3 (50) | M2 (10) | 5520, 1.37 |
| P4 | MA1 (40) | MB1 (50) | M2 (10) | 4676. 1.32 |
| P5* | MA1 (50) | MB2 (50) | | 5932; 1.43 |

*comparative

### Evaluation Results

Photoresist compositions PR-1 to PR-5 were prepared by combining the components indicated in Table 2, with a total solid ratio of 90.91 wt % of the respective polymer and 9.09 wt % of the quencher Q1. The total solids content for each photoresist compositions was 2 wt %. Each mixture was shaken using a mechanical shaker and then filtered through a PTFE disk-shaped filter having a pore size of 0.2 micron.

Evaluation was performed using a CLEAN TRACK ACT8 (TEL, Tokyo Electron Co.) wafer track. 200 nm wafers for photolithographic testing were coated with an AR™ 3 BARC (DuPont Electronics & Industrial) and baked at 205° C. for 60 seconds to give a 60 nm film. A photoresist composition was then coated on the BARC and soft-baked at 110° C. for 60 seconds to give a photoresist film layer having a thickness of about 50 nm.

The wafers were exposed with 248 nm radiation on a CANON FPA-5000 ES4 scanner (NA=0.8, outer sigma=0.85, inner sigma=0.57) with a mask having the features of choice. The wafers were post-exposure baked at 100° C. for 60 seconds, developed with MF™ CD26 TMAH developer (DuPont Electronics & Industrial) for 60 seconds, rinsed with DI water, and dried. Clearing Dose (E₀) values were obtained from an open frame energy meander. Film thickness was measured using a KLA-Tencor Therma-wave OptiProbe ellipsometer. Dose to clear was determined at the energy where resist film thickness reached 20 Å or less.

The structure of the quencher that was used in the formulations and the structure of PAG-A that was used in the comparative example were as follows:

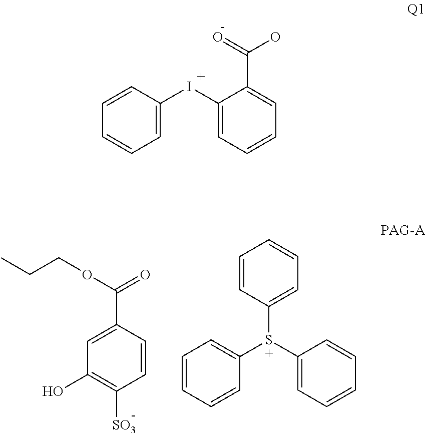

TABLE 2

| Photoresist | | | | E₀ |
|---|---|---|---|---|
| composition | Polymer | PAG | Quencher | (mJ/cm²) |
| PR-1 | P1 (90.9) | — | Q1 (9.1) | 45.0 |
| PR-2 | P2 (90.9) | — | Q1 (9.1) | 34.5 |
| PR-3 | P3 (90.9) | — | Q1 (9.1) | 36.5 |
| PR-4 | P4 (90.9) | — | Q1 (9.1) | 34.5 |
| PR-5* | P5 (73.47) | PAG-A (19.17) | Q1 (7.34) | 26.0 |

*comparative

Table 2 demonstrated that all photoresist compositions from PR-1 to PR-4, which include the inventive polymer-bound photoacid generators (PAGs), show higher dose-to-clear values compared to the comparative example PR-5, which contains a polymer free of PAG and an additive PAG-A. This finding indicates that the incorporation of PAG into the polymer slows acid diffusion.

While this disclosure has been described in connection with what is presently considered to be practical exemplary embodiments, it is to be understood that the invention is not limited to the disclosed embodiments, but, on the contrary, is intended to cover various modifications and equivalent arrangements included within the spirit and scope of the appended claims.

1. A polymerizable compound, comprising:
an anion;
a cation; and
a polymerizable group bonded to the anion or the cation,
wherein the anion comprises: (i) a monocyclic or polycyclic C₃₋₆₀ aromatic group that comprises a first substituent group, and (ii) an anion stabilizing group,
wherein the first substituent group comprises an anion group, wherein the anion group is selected from a sulfonate anion group, a sulfinate anion group, a sulfonimide anion group, a sulfamate anion group, or a sulfonamide anion group,
wherein the anion stabilizing group is configured to form an intramolecular non-covalent bond with the anion group, and
wherein the first substituent group further comprises the anion stabilizing group, or the monocyclic or polycyclic C₃₋₆₀ aromatic group further comprises a second substituent group, wherein the second substituent group comprises the anion stabilizing group.

2. The polymerizable compound of claim 1, wherein the anion is of Formula (1): 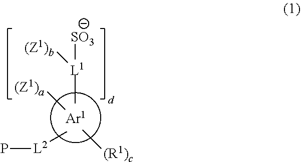
wherein, in Formula (1),
Ar¹ is a monocyclic or polycyclic C₃₋₆₀ aromatic group,
each L¹ is independently a single bond or one or more divalent linking groups, wherein L¹ is free of fluorine,
each L² is independently a single bond or one or more divalent linking groups,
each R¹ is independently a non-hydrogen substituent,
each Z¹ independently comprises an anion stabilizing group, wherein at least one Z¹ is configured to form an intramolecular non-covalent bond with the sulfonate anion group to form a ring having from 5 to 8 atoms, and wherein Z¹ is independently chosen from —OH, —C(O)OH, —SH, —C(O)SH, —NHS(O)₂R², —S(O)₂R², —S(O)₂NHS(O)₂R², —CH(═NOH), or —B(R³)₂; each Z¹ optionally further comprises one or more divalent linking groups as part of its structure,
each R² is independently chosen from fluorine, hydroxyl, substituted or unsubstituted C₁₋₂₀ alkyl, substituted or unsubstituted C₁₋₂₀ heteroalkyl, substituted or unsubstituted C₆₋₃₀ aryl, or substituted or unsubstituted C₃₋₃₀ heteroaryl, and
each R³ is independently chosen from hydrogen, fluorine, hydroxyl, substituted or unsubstituted C₁₋₂₀ alkyl, substituted or unsubstituted C₁₋₂₀ heteroalkyl, substituted or unsubstituted C₆₋₃₀ aryl, or substituted or unsubstituted C₃₋₃₀ heteroaryl,
two R¹ together optionally form a fused ring with Ar¹, wherein the fused ring optionally further comprises one or more divalent linking groups as part of its structure,
one Z¹ and one R¹ together optionally form a fused ring with Ar¹, wherein the fused ring optionally further comprises one or more divalent linking groups as part of its structure,
each a and b is independently an integer from 0 to 2, provided that a sum of a and b is 1 or greater,
c is an integer from 0 to 10,
d is an integer from 1 to 3, and
P is a polymerizable group.

3. The polymerizable compound of claim 2, wherein L² comprises one or more divalent linking groups selected from —O—, —C(O)—, —C(O)O—, —N(R′)—, —C(O)N(R′)—, —S(O)—, —S(O)₂—, substituted or unsubstituted C₁₋₃₀ alkylene, substituted or unsubstituted C₃₋₃₀ cycloalkylene, substituted or unsubstituted C₃₋₃₀ heterocycloalkylene, substituted or unsubstituted C₆₋₃₀ arylene, substituted or unsubstituted C₃₋₃₀ heteroarylene, or a combination thereof, wherein each R′ is independently hydrogen, substituted or unsubstituted C₁₋₂₀ alkyl, substituted or unsubstituted C₁₋₂₀ heteroalkyl, substituted or unsubstituted C₆₋₃₀ aryl, or substituted or unsubstituted C₃₋₃₀ heteroaryl.

4. The polymerizable compound of claim 2, wherein L¹ is a single bond.

5. The polymerizable compound of claim 1, wherein the cation is iodonium or sulfonium.

6. The polymerizable compound of claim 1, wherein the cation comprises ammonium.

7. The polymerizable compound of claim 1, wherein at least one anion stabilizing group comprises —OH.

8. The polymerizable compound of claim 1, wherein Ar¹ is a monocyclic C₆ aromatic group.

9. The polymerizable compound of claim 1, wherein L² comprises an iodo-substituted aromatic group.

10. A photoresist composition, comprising:
the polymerizable compound of claim 1; or a polymer comprising a repeating unit derived from the polymerizable compound of claim 1; and
a solvent.

11. The photoresist composition of claim 10, further comprising a non-solvent alkali-insoluble base material.

12. A patterning method, the method comprising:
applying a layer of the photoresist composition of claim 10 on a substrate to provide a photoresist composition layer;
pattern-wise exposing the photoresist composition layer to activating radiation to provide an exposed photoresist composition layer; and
developing the exposed photoresist composition layer to provide a resist relief image.

13. The photoresist composition of claim 10, wherein the anion is of Formula (1): 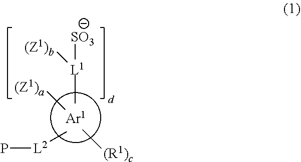
wherein, in Formula (1),
Ar¹ is a monocyclic or polycyclic C₃₋₆₀ aromatic group,
each L¹ is independently a single bond or one or more divalent linking groups, wherein L¹ is free of fluorine,
each L² is independently a single bond or one or more divalent linking groups,
each R¹ is independently a non-hydrogen substituent,
each Z¹ independently comprises an anion stabilizing group, wherein at least one Z¹ is configured to form an intramolecular non-covalent bond with the sulfonate anion group to form a ring having from 5 to 8 atoms, and wherein Z¹ is independently chosen
from —OH, —C(O)OH, —SH, —C(O)SH, —NHS(O)₂R², —S(O)₂R², —S(O)₂NHS(O)₂R², —CH(═NOH), or —B(R³)₂; each Z¹ optionally further comprises one or more divalent linking groups as part of its structure,
each R² is independently chosen from fluorine, hydroxyl, substituted or unsubstituted C₁₋₂₀ alkyl, substituted or unsubstituted C₁₋₂₀ heteroalkyl, substituted or unsubstituted C₆₋₃₀ aryl, or substituted or unsubstituted C₃₋₃₀ heteroaryl, and
each R³ is independently chosen from hydrogen, fluorine, hydroxyl, substituted or unsubstituted C₁₋₂₀ alkyl, substituted or unsubstituted C₁₋₂₀ heteroalkyl, substituted or unsubstituted C₆₋₃₀ aryl, or substituted or unsubstituted C₃₋₃₀ heteroaryl,
two R¹ together optionally form a fused ring with Ar¹, wherein the fused ring optionally further comprises one or more divalent linking groups as part of its structure,
one Z¹ and one R¹ together optionally form a fused ring with Ar¹, wherein the fused ring optionally further comprises one or more divalent linking groups as part of its structure,
each a and b is independently an integer from 0 to 2, provided that a sum of a and b is 1 or greater,
c is an integer from 0 to 10,
d is an integer from 1 to 3, and
P is a polymerizable group.

14. The photoresist composition of claim 10, wherein L² comprises one or more divalent linking groups selected from —O—, —C(O)—, —C(O)O—, —N(R′)—, —C(O)N(R′)—, —S(O)—, —S(O)₂—, substituted or unsubstituted C₁₋₃₀ alkylene, substituted or unsubstituted C₃₋₃₀ cycloalkylene, substituted or unsubstituted C₃₋₃₀ heterocycloalkylene, substituted or unsubstituted C₆₋₃₀ arylene, substituted or unsubstituted C₃₋₃₀ heteroarylene, or a combination thereof, wherein each R′ is independently hydrogen, substituted or unsubstituted C₁₋₂₀ alkyl, substituted or unsubstituted C₁₋₂₀ heteroalkyl, substituted or unsubstituted C₆₋₃₀ aryl, or substituted or unsubstituted C₃₋₃₀ heteroaryl.

15. The photoresist composition of claim 10, wherein L¹ is a single bond.

16. The photoresist composition of claim 10, wherein the cation is iodonium or sulfonium.

17. The photoresist composition of claim 10, wherein the cation comprises ammonium.

18. The photoresist composition of claim 10, wherein at least one anion stabilizing group comprises —OH.

19. The photoresist composition of claim 10, wherein Ar¹ is a monocyclic C₆ aromatic group.

20. The photoresist composition of claim 10, wherein L² comprises an iodo-substituted aromatic group.
